SEQUENCE LISTING (117 sequences)

SEQ ID NO: 1 - skipped

SEQ ID NO: 2 - protein, 10 aa, synthetic construct
GGGSSGGGSG

SEQ ID NO: 3 - protein, 12 aa, synthetic construct
ESKYGPPCPPCP

SEQ ID NO: 4 - protein, 12 aa, synthetic construct
ESKYGPPCPSCP

SEQ ID NO: 5 - protein, 22 aa, synthetic construct
ESKYGPPCPPCPGGGSSGGGSG

SEQ ID NO: 6 - protein, 39 aa, Homo sapiens
IEVMYPPPYLDNEKSNGTIIHVKGKHLCPSPLFPGPSKP

SEQ ID NO: 7 - protein, 48 aa, Homo sapiens
AKPTTTPAPRPPTPAPTIASQPLSLRPEACRPAAGGAVHTRGLDFACD

SEQ ID NO: 8 - protein, 45 aa, Homo sapiens
TTTPAPRPPTPAPTIASQPLSLRPEACRPAAGGAVHTRGLDFACD

SEQ ID NO: 9 - protein, 129 aa, synthetic construct
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 229 aa, synthetic construct
ESKYGPPCPSCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 229 aa, synthetic construct
ESKYGPPCPPCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 21 aa, Homo sapiens
LCYLLDGILFIYGVILTALFL

SEQ ID NO: 14 - protein, 27 aa, Homo sapiens
FWVLVVVGGVLACYSLLVTVAFIIFWV

SEQ ID NO: 15 - protein, 28 aa, Homo sapiens
MFWVLVVVGGVLACYSLLVTVAFIIFWV

SEQ ID NO: 16 - protein, 22 aa, Homo sapiens
MALIVLGGVAGLLLFIGLGIFF

SEQ ID NO: 17 - protein, 21 aa, Homo sapiens
IYIWAPLAGTCGVLLLSLVIT

SEQ ID NO: 18 - protein, 23 aa, Homo sapiens
IYIWAPLAGTCGVLLLSLVITLY

SEQ ID NO: 19 - protein, 24 aa, Homo sapiens
IYIWAPLAGTCGVLLLSLVITLYC

SEQ ID NO: 20 - protein, 27 aa, Homo sapiens
IISFFLALTSTALLFLLFFLTLRFSVV

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 41 aa, Homo sapiens
RSKRSRLLHSDYMNMTPRRPGPTRKHYQPYAPPRDFAAYRS

SEQ ID NO: 23 - protein, 41 aa, Homo sapiens
RSKRSRGGHSDYMNMTPRRPGPTRKHYQPYAPPRDFAAYRS

SEQ ID NO: 24 - protein, 42 aa, Homo sapiens
KRGRKKLLYIFKQPFMRPVQTTQEEDGCSCRFPEEEEGGCEL

SEQ ID NO: 25 - protein, 42 aa, Homo sapiens
ALYLLRRDQRLPPDAHKPPGGGSFRTPIQEEQADAHSTLAKI

SEQ ID NO: 26 - protein, 22 aa, Homo sapiens
MLLLVTSLLLCELPHPAFLLIP

SEQ ID NO: 27 - protein, 24 aa, synthetic construct
LEGGGEGRGSLLTCGDVEENPGPR

SEQ ID NO: 28 - protein, 354 aa, synthetic construct
LVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 29 - protein, 576 aa, synthetic construct
QVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPR

SEQ ID NO: 30 - protein, 676 aa, synthetic construct
QVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPR

SEQ ID NO: 31 - protein, 465 aa, synthetic construct
QVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKESKYGPPCPPCPMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPR

SEQ ID NO: 32 - protein, 576 aa, synthetic construct
QVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQYGSLAWYQQRPGQAPRLVIYSGSTRAAGIPDRFSGSRWGADYNLSISNLESGDFGVYYCQQYEFFGQGTKVQVDIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPR

SEQ ID NO: 33 - protein, 465 aa, synthetic construct
QVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQYGSLAWYQQRPGQAPRLVIYSGSTRAAGIPDRFSGSRWGADYNLSISNLESGDFGVYYCQQYEFFGQGTKVQVDIKESKYGPPCPPCPMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPR

SEQ ID NO: 34 - protein, 676 aa, synthetic construct
QVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQYGSLAWYQQRPGQAPRLVIYSGSTRAAGIPDRFSGSRWGADYNLSISNLESGDFGVYYCQQYEFFGQGTKVQVDIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPR

SEQ ID NO: 35 - protein, 576 aa, synthetic construct
QVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPR

SEQ ID NO: 36 - protein, 676 aa, synthetic construct
QVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPR

SEQ ID NO: 37 - protein, 465 aa, synthetic construct
QVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKESKYGPPCPPCPMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPR

SEQ ID NO: 38 - protein, 573 aa, synthetic construct
RAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGSGGGSGGGSGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPR

SEQ ID NO: 39 - protein, 673 aa, synthetic construct
RAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGSGGGSGGGSGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPR

SEQ ID NO: 40 - protein, 462 aa, synthetic construct
RAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGSGGGSGGGSGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKESKYGPPCPPCPMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPR

SEQ ID NO: 41 - protein, 955 aa, synthetic construct
MLLLVTSLLLCELPHPAFLLIPQVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 42 - protein, 1055 aa, synthetic construct
MLLLVTSLLLCELPHPAFLLIPQVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 43 - protein, 844 aa, synthetic construct
MLLLVTSLLLCELPHPAFLLIPQVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKESKYGPPCPPCPMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 44 - protein, 955 aa, synthetic construct
MLLLVTSLLLCELPHPAFLLIPQVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQYGSLAWYQQRPGQAPRLVIYSGSTRAAGIPDRFSGSRWGADYNLSISNLESGDFGVYYCQQYEFFGQGTKVQVDIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 45 - protein, 844 aa, synthetic construct
MLLLVTSLLLCELPHPAFLLIPQVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQYGSLAWYQQRPGQAPRLVIYSGSTRAAGIPDRFSGSRWGADYNLSISNLESGDFGVYYCQQYEFFGQGTKVQVDIKESKYGPPCPPCPMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 46 - protein, 1055 aa, synthetic construct
MLLLVTSLLLCELPHPAFLLIPQVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQYGSLAWYQQRPGQAPRLVIYSGSTRAAGIPDRFSGSRWGADYNLSISNLESGDFGVYYCQQYEFFGQGTKVQVDIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 47 - protein, 955 aa, synthetic construct
MLLLVTSLLLCELPHPAFLLIPQVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 48 - protein, 1055 aa, synthetic construct
MLLLVTSLLLCELPHPAFLLIPQVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 49 - protein, 844 aa, synthetic construct
MLLLVTSLLLCELPHPAFLLIPQVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKESKYGPPCPPCPMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 50 - protein, 952 aa, synthetic construct
MLLLVTSLLLCELPHPAFLLIPRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGSGGGSGGGSGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 51 - protein, 1052 aa, synthetic construct
MLLLVTSLLLCELPHPAFLLIPRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGSGGGSGGGSGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMALIVLGGVAGLLLFIGLGIFFKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 52 - protein, 841 aa, synthetic construct
MLLLVTSLLLCELPHPAFLLIPRAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGSGGGSGGGSGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIKESKYGPPCPPCPMFWVLVVVGGVLACYSLLVTVAFIIFWVKRGRKKLLYIFKQPFMRPVQTTQEEDGCSCRFPEEEEGGCELGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 53 - skipped

SEQ ID NO: 54 - protein, 101 aa, synthetic construct
EIVLTQSPATLSLSPGETAIISCRTSQSGSLAWYQQRPGQAPRLVIYSGSTRAAGIPDRFSRWGADYNLSISNLESGDFGVYYCQQYEFFGQGTKVQVDIK

SEQ ID NO: 55 - protein, 105 aa, synthetic construct
SDISVAPGETARISCGEKSLGSRAVQWYQHRAGQAPSLIIYNNQDRPSGIPERFSGSPDSPFGTTATLTITSVEAGDEADYYCHIWDSRVPTKWVFGGGTTLTVL

SEQ ID NO: 56 - protein, 114 aa, synthetic construct
DFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKRT

SEQ ID NO: 57 - protein, 110 aa, synthetic construct
QSVLTQPASVSASPGQSITVSCTGSRNDVGGYDFVSWYQRHPGGVPKLIIYEISKRPSGIPQRFSGSRSGNTASLTISGLQDDDEADYYCCSYASYDRLIFGGGTRVSVL

SEQ ID NO: 58 - protein, 105 aa, synthetic construct
QSALTQPPSASGSPGQSISISCTGTSNRFVSWYQQHPGKAPKLVIYGVNKRPSGVPDRFSGSKSGNTASLTVSGLQTDDEAVYYCSSLVGNWDVIFGGGTKLTVL

SEQ ID NO: 59 - protein, 105 aa, synthetic construct
QSALTQPPSASGSPGQSITISCTGTSNNFVSWYQQHAGKAPKLVIYDVNKRPSGVPDRFSGSKSGNTASLTVSGLQTDDEAVYYCGSLVGNWDVIFGGGTKLTVL

SEQ ID NO: 60 - protein, 114 aa, synthetic construct
EVVITQSPLFLPVTPGEAASLSCKCSHSLQHSTGANYLAWYLQRPGQTPRLLIHLATHRASGVPDRFSGSGSGTDFTLKISRVESDDVGTYYCMQGLHSPWTFGQGTKVEIKRT

SEQ ID NO: 61 - protein, 110 aa, synthetic construct
QSALTQPASVSGSPGQSITISCNGTSNDVGGYESVSWYQQHPGKAPKVVIYDVSKRPSGVSNRFSGSKSGNTASLTISGLQAEDEGDYYCKSLTSTRRRVFGTGTKLTVL

SEQ ID NO: 62 - protein, 110 aa, synthetic construct
QSALTQPASVSGSPGQTITISCNGTSSDVGGFDSVSWYQQSPGKAPKVMVFDVSHRPSGISNRFSGSKSGNTASLTISGLHIEDEGDYFCSSLTDRSHRIFGGGTKVTVL

SEQ ID NO: 63 - protein, 104 aa, synthetic construct
EIVLTQSPGTLSLSPGETAIISCRISQYGSLAWYQQRPGQAPRLVIYSGSTRAAGIPDRFSGSRWGPDYNLTISNLESGDFGVYYCQQYEFFGQGTKVQVDIKR

SEQ ID NO: 64 - protein, 103 aa, synthetic construct
SLTQSPGTLSLSPGETAIISCRTSQYGSLAWYQQRPGQAPRLVIYSGSTRAATGIPDRFSGSRWGPDYNLTISNLESGDFGVYYCQQYEFFGQGTKVQVDIKR

SEQ ID NO: 65 - protein, 107 aa, synthetic construct
ELVLTQSPGTLSLSAGERATLSCRASQSVSSGSLAWYQQKPGQAPRLLIYGASTRATGIPDRFSGSGSGTDFILTIGRLEPEDLAVYYCQQYGTSPYTFGQGTKLEI

SEQ ID NO: 66 - protein, 109 aa, synthetic construct
QSVLTQSASVSGSLGQSVTISCTGPNSVCCSHKSISWYQWPPGRAPTLIIYEDNERAPGISPRFSGYKSYWSAYLTISDLRPEDETTYYCCSYTHNSGCVFGTGTKVSV

SEQ ID NO: 67 - protein, 108 aa, synthetic construct
SYELTQETGVSVALGRTVTITCRGDSLRSHYASWYQKKPGQAPILLFYGKNNRPSGVPDRFSGASGNRASLTISGAQAEDDAEYYCSSRDKSGSRLSVFGGGTKLTVL

SEQ ID NO: 68 - protein, 105 aa, synthetic construct
DIQMTQSPSSLSASVGDTVTITCQANGYLNWYQQRRGKAPKLLIYDGSKLERGVPSRFSGRRWGQEYNLTINNLQPEDIATYFCQVYEFVVPGTRLDLKRTVAAP

SEQ ID NO: 69 - protein, 104 aa, synthetic construct
SEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIK

SEQ ID NO: 70 - protein, 104 aa, synthetic construct
SEIVLTQSPATLSLSPGETAIISCRTSQYGSLAWYQQRPGQAPRLVIYSGSTRAAGIPDRFSGSRWGADYNLSISNLESGDFGVYYCQQYEFFGQGTKVQVDIK

SEQ ID NO: 71 - skipped

SEQ ID NO: 72 - protein, 244 aa, synthetic construct
QVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIK

SEQ ID NO: 73 - protein, 22 aa, synthetic construct
MALIVLGGVAGLLLFIGLGIFF

SEQ ID NO: 74 - protein, 24 aa, synthetic construct
LEGGGEGRGSLLTCGDVEENPGPR

SEQ ID NO: 75 - protein, 335 aa, synthetic construct
RKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 76 - skipped

SEQ ID NO: 77 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78-79 - skipped (2 entries)

SEQ ID NO: 80 - protein, 244 aa, synthetic construct
QVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQYGSLAWYQQRPGQAPRLVIYSGSTRAAGIPDRFSGSRWGADYNLSISNLESGDFGVYYCQQYEFFGQGTKVQVDIK

SEQ ID NO: 81-83 - skipped (3 entries)

SEQ ID NO: 84 - protein, 245 aa, synthetic construct
QVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIKE

SEQ ID NO: 85 - protein, 11 aa, synthetic construct
SKYGPPCPPCP

SEQ ID NO: 86 - skipped

SEQ ID NO: 87 - protein, 244 aa, synthetic construct
QVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSSGGGSGGGSGGGSGGGSEIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIK

SEQ ID NO: 88-89 - skipped (2 entries)

SEQ ID NO: 90 - protein, 241 aa, synthetic construct
RAHLVQSGTAMKKPGASVRVSCQTSGYTFTAHILFWFRQAPGRGLEWVGWIKPQYGAVNFGGGFRDRVTLTRDVYREIAYMDIRGLKPDDTAVYYCARDRSYGDSSWALDAWGQGTTVVVSAGGGSGGGSGGGSGGGSYIHVTQSPSSLSVSIGDRVTINCQTSQGVGSDLHWYQHKPGRAPKLLIHHTSSVEDGVPSRFSGSGFHTSFNLTISDLQADDIATYYCQVLQFFGRGSRLHIK

SEQ ID NO: 91-92 - skipped (2 entries)

SEQ ID NO: 93 - protein, 103 aa, synthetic construct
EIVLTQSPATLSLSPGETAIISCRTSQSGSLAWYQQRPGQAPRLVIYSGSTRAAGIPDRFSGSRWGADYNLSISNLESGDFGVYYCQQYEFFGQGTKVQVDIK

SEQ ID NO: 94 - protein, 125 aa, synthetic construct
QVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSS

SEQ ID NO: 95 - protein, 103 aa, synthetic construct; identical to SEQ ID NO: 93

SEQ ID NO: 96 - protein, 125 aa, synthetic construct
QVRLSQSGGQMKKPGESMRLSCRASGYEFLNCPINWIRLAPGRRPEWMGWLKPRWGAVNYARKFQGRVTMTRDVYSDTAFLELRSLTSDDTAVYFCTRGKYCTARDYYNWDFEHWGRGAPVTVSS

SEQ ID NO: 97 - protein, 103 aa, synthetic construct
EIVLTQSPATLSLSPGETAIISCRTSQYGSLAWYQQRPGQAPRLVIYSGSTRAAGIPDRFSGSRWGADYNLSISNLESGDFGVYYCQQYEFFGQGTKVQVDIK

SEQ ID NO: 98 - protein, 125 aa, synthetic construct; identical to SEQ ID NO: 96

SEQ ID NO: 99 - protein, 103 aa, synthetic construct [sequence omitted]

SEQ ID NO: 100 - protein, 122 aa, synthetic construct [sequence omitted]

SEQ ID NO: 101 - protein, 147 aa, synthetic construct
MLLLVTSLLLCELPHPAFLLIPQVRLSQSGGQMKKPGESMRLSCRASGYEFLNCPINWIRLAPGRRPEWMGWLKPRGGAVNYARKFQGRVTMTRDVYSDTAFLELRSLTSDDTAVYFCTRGKYCTARDYYNWDFEHWGRGAPVTVSS

SEQ ID NO: 102 - protein, 154 aa, synthetic construct
MLLLVTSLLLCELPHPAFLLIPQMQLQESGPGLVKPSETLSLTCSVSGASISDSYWSWIRRSPGKGLEWIGYVHKSGDTNYSPSLKSRVNLSLDTSKNQVSLSLVAATAADSGKYYCARTLHGRRIYGIVAFNEWFTYFYMDVWGNGTQVTVSS

SEQ ID NO: 103 - protein, 163 aa, synthetic construct
MLLLVTSLLLCELPHPAFLLIPQAQLVQSGPEVRKPGTSVKVSCKAPGNTLKTYDLHWVRSVPGQGLQWMGWISHEGDKKVIVERFKAKVTIDWDRSTNTAYLQLSGLTSGDTAVYYCAKGSKHRLRDYALYDDDGALNWAVDVDYLSNLEFWGQGTAVTVSS

SEQ ID NO: 104 - protein, 150 aa, synthetic construct
MLLLVTSLLLCELPHPAFLLIPQVQLMQSGAQLRDPGDSLKISCKASGYNFIDYHIHWVRLAPGRGLEWMGWIDPVGGITKYAGQFQGRLSLTRDTSTNTLFLELSRLTAGDTAVYFCARSMRPVDHGIDYSGLFVFHFWGRGSDVLVSS

SEQ ID NO: 105 - protein, 159 aa, synthetic construct
MLLLVTSLLLCELPHPAFLLIPQPQLQESGPGLVEASETLSLTCTVSGDSTAACDYFWGWVRQPPGKGLEWIGGLSHCAGYYNTGWTYHNPSLKSRLTISLDTPKNQVFLKLNSVTAADTAIYYCARFDGEVLVYHDWPKPAWVDLWGRGTLVTVTVSS

SEQ ID NO: 106 - protein, 157 aa, synthetic construct
MLLLVTSLLLCELPHPAFLLIPQPQLQESGPTLVEASETLSLTCAVSGDSTAACNSFWGWVRQPPGKGLEWVGSLSHCASYWNRGWTYHNPSLKSRLTLALDTPKNLVFLKLNSVTAADTATYYCARFGGEVLRYTDWPKPAWVDLWGRGTLVTVSS

SEQ ID NO: 107 - protein, 162 aa, synthetic construct
MLLLVTSLLLCELPHPAFLLIPQVQLVQSGAEVKKPGSSVKVSCKASGNSFSNHDVHWVRQATGQGLEWMGWMSHEGDKTGLAQKFQGRVTITRDSGASTVYMELRGLTADDTAIYYCLTGSKHRLRDYFLYNEYGPNYEEWGDYLATLDVWGHGTAVTVSS

SEQ ID NO: 108 - protein, 157 aa, synthetic construct
MLLLVTSLLLCELPHPAFLLIPQRLVESGGGVVQPGSSLRSCAASGFDFSRQGMHWVRQAPGQGLEWVAFIKYDGSEKYHADSVWGRLSISRDNSKDTLYLQMNSLRVEDTATYFCVREAGGPDYRNGYNYYDFYDGYYNYHYMDVWGKGTTVTVSS

SEQ ID NO: 109 - protein, 159 aa, synthetic construct
MLLLVTSLLLCELPHPAFLLIPQEQLVESGGGVVQPGGSLRLSCLASGFTFHKYGMHWVRQAPGKGLEWVALISDDGMRKYHSDSMWGRVTISRDNSKNTLYLQFSSLKVEDTAMFFCAREAGGPIWHDDVKYYDFNDGYYNYHYMDVWGKGTTVTVSS

SEQ ID NO: 110 - protein, 143 aa, synthetic construct
MLLLVTSLLLCELPHPAFLLIPQVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSS

SEQ ID NO: 111 - protein, 147 aa, synthetic construct
MLLLVTSLLLCELPHPAFLLIPQVRLSQSGGQMKKPGDSMRISCRASGYEFINCPINWIRLAPGKRPEWMGWMKPRHGAVSYARQLQGRVTMTRDMYSETAFLELRSLTSDDTAVYFCTRGKYCTARDYYNWDFEHWGQGTPVTVSS

SEQ ID NO: 112 - protein, 150 aa, synthetic construct
MLLLVTSLLLCELPHPAFLLIPLEQSGAEVKKPGSSVQVSCKASGGTFSMYGFNWVRQAPGHGLEWMGGIIPIFGTSNYAQKFRGRVTFTADQATSTAYMELTNLRSDDTAVYYCARDFGPDWEDGDSYDGSGRGFFDFWGQGTLVTVSS

SEQ ID NO: 113 - protein, 153 aa, synthetic construct
MLLLVTSLLLCELPHPAFLLIPQGQLVQSGAELKKPGASVKISCKTSGYRFNFYHINWIRQTAGRGPEWMGWISPYSGDKNLAPAFQDRVIMTTDTEVPVTSFTSTGAAYMEIRNLKFDDTGTYFCAKGLLRDGSSTWLPYLWGQGTLLTVSS

SEQ ID NO: 114 - protein, 153 aa, synthetic construct
MLLLVTSLLLCELPHPAFLLIPEVQLVESGGGLVKPGGSLRLSCSASGFDFDNAWMTWVRQPPGKGLEWVGRITGPGEGWSVDYAAPVEGRFTISRLNSINFLYLEMNNLRMEDSGLYFCARTGKYYDFWSGYPPGEEYFQDWGRGTLVTVSS

SEQ ID NO: 115 - protein, 151 aa, synthetic construct
MLLLVTSLLLCELPHPAFLLIPQVQLLQSGAAVTKPGASVRVSCEASGYNIRDYFIHWWRQAPGQGLQWVGWINPKTGQPNNPRQFQGRVSLTRHASWDFDTFSFYMDLKALRSDDTAVYFCARQRSDYWDFDVWGSGTQVTVSSASTKGP

SEQ ID NO: 116 - protein, 125 aa, synthetic construct; identical to SEQ ID NO: 96

SEQ ID NO: 117 - protein, 473 aa, synthetic construct
MVRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASGGGGSGGGGSQVQLLESGAEVKKPGSSVKVSCKASGDTFIRYSFTWVRQAPGQGLEWMGRIITILDVAHYAPHLQGRVTITADKSTSTVYLELRNLRSDDTAVYFCAGVYEGEADEGEYDNNGFLKGWGQGTLVTVTSGGGGSGGGGSGGGGSELELTQSPATLSVSPGERATLSCRASESVSSDLAWYQQKPGQAPRLLIYGASTRATGVPARFSGSGSGAEFTLTISSLQSEDFAVYYCQQYNNWPPRYTFGQGTRLEIKAAA

(19) United States
(12) Patent Application Publication
(10) Pub. No.: US 2026/0048117 A1
(43) Pub. Date: Feb. 19, 2026

(54) CAR for Treatment of HIV Infection

(71) Applicant: City of Hope, Duarte, CA (US)

(72) Inventors: John Zaia, Duarte, CA (US); Kevin Morris, Duarte, CA (US); Xiuli Wang, Temple City, CA (US); Stephen J. Forman, Duarte, CA (US)

(21) Appl. No.: 19/379,100

(22) Filed: Nov. 4, 2025

Related U.S. Application Data
(63) Continuation of application No. 16/758,516, now Pat. No. 12,496,338.
(60) Provisional application No. 62/576,063, filed on Oct. 23, 2017.

(51) Int. Cl. A61K 39/245; A61K 40/11; A61K 40/31; A61K 40/46; A61P 31/18; C07K 14/725; C07K 16/10; C12N 5/0783

(52) U.S. Cl. CPC A61K 39/245; A61K 40/11; A61K 40/31; A61K 40/46; A61P 31/18; C07K 14/7051; C07K 16/1063; C12N 5/0638; A61K 2239/38

(57) ABSTRACT

Methods of treatment for HIV employing T cells expressing both a chimeric antigen receptors targeted to HIV and a CMV-specific T cell receptor.

### CROSS-REFERENCE TO RELATED APPLICATIONS

This application is a continuation of U.S. patent Ser. No. 16/758,516, filed on Feb. 23, 2020, which is the National Stage Application of International Application No. PCT/US2018/057124, filed on Oct. 23, 2018, which claims priority to U.S. Application No. 62/576,063, filed on Oct. 23, 2017. The entire contents of the foregoing are incorporated herein by reference.

### FEDERALLY SPONSORED RESEARCH OR DEVELOPMENT

This invention was made with government support under MH113407, AI099783, AI111139, and DK104681 awarded by the National Institutes of Health. The government has certain rights in the invention

### SEQUENCE LISTING

This application contains a Sequence Listing that has been submitted electronically as an XML file named “40056-0036002_SL_ST26.XML.” The XML file, created on Nov. 4, 2025, is 119,484 bytes in size. The material in the XML file is hereby incorporated by reference in its entirety.

### BACKGROUND

HIV-infected individuals can develop a T cell response to HIV. However, the virus can largely evade endogenous T cell-mediated immune response through various mechanisms and, in the vast majority of cases, this loss of T cell function is critical for continued HIV replication. Thus, antiretroviral therapy (ART), which can dramatically reduce HIV replication, is currently the only method for control of HIV and has become the standard of care. Because ART does not eliminate the virus, people living with HIV must remain on ART therapy indefinitely. Accordingly, it has been suggested that there is a new way to develop an engineered immune response to provide long-lasting control of HIV infection in the absence of ART.

### SUMMARY

Described herein are methods for reducing or eliminating HIV-infected cells, including latently infected cells, in an HIV-infected individual using chimeric antigen receptor (CAR) T cell therapy. The methods use a CMV-specific T cell platform for making CAR T cells that are redirected to kill HIV-infected cells and are responsive to a CMV vaccine to maintain and boost levels and activity of these immune cells. This immunotherapy, described herein, kills HIV-infected cells in a manner that also limits the emergence of viral escape, ultimately resulting in reduction or eradication of HIV-infected cells. The approach entails generating CMV-HIV CAR T cells and infusing these modified T cells into HIV subjects, either before, during, or after they have had their antiretroviral therapy (ART) removed (FIG. 1). In some cases, CMV-HIV CAR T cells targeted to two or more (e.g., four) distinct epitopes on the HIV gp 120 membrane protein can be used (FIGS. 3A-3B).

In various embodiments: the chimeric antigen receptor comprises: a single-chain variable fragment (scFv) of an anti-HIV-1 gp120 antibody selected from those in FIGS. 3A-3B; a spacer region; a CD28 transmembrane domain; a CD28 co-signaling domain; and a CD3 ζ signaling domain; the chimeric antigen receptor comprises: a HIV scFv; a spacer region; a CD4 transmembrane domain; a 4-1BB co-signaling domain; and a CD3 ζ signaling domain; the chimeric antigen receptor comprises: a HIV scFv; a spacer region comprising an amino acid sequence selected from SEQ ID Nos:2-5 and 9-12; a CD4 transmembrane domain; a 4-1BB co-signaling domain; and a CD3 ζ signaling domain; the chimeric antigen receptor comprises: a HIV scFv; a spacer region comprising an amino acid sequence selected from SEQ ID Nos:2-5 and 9-12; a CD28 transmembrane domain; a CD28 co-signaling domain; and a CD3 ζ signaling domain; the chimeric antigen receptor comprises: a HIV scFv; a spacer a spacer comprising an amino acid sequence selected from SEQ ID Nos:2-5 and 9-12; CD4 transmembrane domain; a 4-1BB co-signaling domain; and CD3 ζ signaling domain; the chimeric antigen receptor comprises: a spacer a spacer comprising an amino acid sequence selected from SEQ ID Nos:2-5 and 9-12; a CD28 transmembrane domain; a CD28 co-signaling domain; and a CD3 ζ signaling domain; the chimeric antigen receptor comprises an amino acid sequence at least 95% identical to an amino acid sequence selected from: SEQ ID NOs: 20-40; the chimeric antigen receptor comprises an amino acid sequence identical to an amino acid sequence selected from: SEQ ID NOs: 29-30; the chimeric antigen receptor comprises an amino acid sequence identical to an amino acid sequence selected from: SEQ ID NOs: 29-40, each with no more than 5 single amino acid substitutions; at least 20%, 30%, or 40% of the transduced human T cells are central memory T cells; at least 30% of the transduced human T cells are CD4+ and CD62L+ or CD8+ and CD62L+; the population of human T cells are autologous to the patient; and the population of human T cells are allogenic to the patient. Described herein is a human T cell expressing: (a) a T cell receptor (TCR) that is activated by binding to a CMV antigen; and (b) a chimeric antigen receptor (CAR) comprising an antigen-binding domain that binds to an epitope on HIV gp120, a spacer, a transmembrane domain, a co-stimulatory domain, and a CD3 zeta signaling domain. In various embodiments: the antigen-binding domain is a scFv comprising a heavy chain variable region and a light chain variable region; the heavy chain variable region has an amino acid sequence as shown in FIGS. 5A-5B, and wherein the light chain variable region has an amino acid sequence that is the corresponding light chain variable region sequence shown in FIGS. 5A-5B; wherein the transmembrane domain is a CD4, CD8 or CD28 transmembrane domain; wherein the co-stimulatory domain is a 4-1BB or CD28 co-stimulatory domain; the cell expresses a second CAR wherein the first CAR comprises a first antigen-binding domain, a first spacer, a first transmembrane domain, a first co-stimulatory domain, and a first intracellular signaling domain, wherein the second CAR comprises a second antigen-binding domain, a second spacer, a second transmembrane domain, a second co-stimulatory domain, and a second intracellular signaling domain, wherein the first antigen-binding domain is different from the second antigen-binding domain, and wherein the first antigen-binding domain binds to a first epitope on HIV gp120, and the second antigen-binding domain binds to a second epitope on HIV gp120, wherein the second epitope is the same as or different from the first epitope; and the first antigen-binding domain comprises a first scFv, and the second antigen-binding domain comprises a second scFv, wherein the amino acid sequence of the first scFv is different from the amino acid sequence of the second scFv.

Also described is a method for treating HIV comprising administering to a patient in need thereof a population of human T cells comprising T cells expressing CAR targeted to two or more HIV gp120 epitopes. In various embodiments: the chimeric antigen receptor comprises: a HIV scFv; a spacer region; a CD4, CD8 or CD28 transmembrane domain; a 4-IBB of CD28 co-signaling domain; and a CD3 ζ signaling domain; the T cells express a T cell receptor specific for a cytomegalovirus (CMV) antigen; the population of human T cells comprising T cells expressing CAR targeted to three or more HIV gp120 epitopes; the population of human T cells comprising T cells expressing CAR targeted to four or more HIV gp120 epitopes; the population of human T cells comprising T cells expressing CAR targeted to six or more HIV gp120 epitopes; the patient is not being administered anti-retroviral therapy at the time of administration; the patient is further administered a T cell antigen; the chimeric antigen receptor comprises an amino acid sequence identical to an amino acid sequence selected from: SEQ ID NOs: 29-40 or 41-53; the method further comprises: administering to the patient a viral vector encoding: (i) CMV pp65 and (ii) a fusion protein comprising exon 4 of CMV protein IE1 (e4) and exon 5 of CMV protein IE2 (e5) either prior to or subsequent to administering the composition comprising a population of T cells to the patient; the T cells are allogenic to the patient; T cells are engineered autologous T cells; the patient is treated to activate HIV-1 provirus before or after administration of the population of T cells; the T cells express a T cell receptor specific for a cytomegalovirus (CMV) antigen and are expanded in the presence of a T cell antigen before administration to the patient; the CMV antigen is a CMV peptide or a CMV vaccine; the expansion is in the presence of any HIV antigen and a CMV antigen; and the expansion is in the absence of an HIV antigen.

Also described is a method of treating HIV in an HIV infected subject, comprising: obtaining a population of cells comprising PBMC from the subject and treating the cells to obtain a subpopulation of T cells that express a TCR that binds to an CMV antigen; treating the subpopulation of cells to introduce a vector encoding a CAR that binds to an epitope on HIV gp120, thereby creating a population of bi-specific T cells expressing a TCR that binds to a CMV antigen and a CAR that binds to an epitope on HIV gp120; expanding the population of bi-specific T cells; and administering the expanded bi-specific T cells to the subject.

In various embodiments: the method further comprises administering a CMV antigen to the subject prior to obtaining a population of cells comprising PBMC from the subject; a CMV antigen is administered to the patient before, in conjunction with or after administering the expanded bi-specific T cells to the subject; the CAR comprises an antigen-binding domain that binds to an epitope on HIV gp120, a spacer, a transmembrane domain, a co-stimulatory domain, and a CD3 zeta signaling domain; the antigen-binding domain is a scFv comprising a heavy chain variable region and a light chain variable region; the heavy chain variable region has an amino acid sequence as shown in FIGS. 5A-5B, and wherein the light chain variable region has an amino acid sequence that is the corresponding light chain variable region sequence shown in FIGS. 5A-5B; the transmembrane domain is a CD4, CD8 or CD28 transmembrane domain; the co-stimulatory domain is a 4-1BB or CD28 co-stimulatory domain; the CAR comprises the amino acid sequence of any of SEQ ID NOS: 20-50 with up to 10 single amino acids substitutions; the CAR comprises the amino acid sequence of any of SEQ ID NOS: 20-50 with up to 5 single amino acids substitutions; the CAR comprises the amino acid sequence of any of SEQ ID NOS: 20-50 with up to 10 single amino acids substitutions; the T cells express a T cell receptor specific for a cytomegalovirus (CMV) antigen and are expanded in the presence of a T cell antigen before administration to the patient; the CMV antigen is a CMV peptide or a CMV vaccine; the expansion is in the presence of a HIV antigen and a CMV antigen; and the expansion is in the absence of an HIV antigen.

Also described is a method comprising: obtaining a population of PBMC comprising T cells express a TCR that binds to a CMV antigen; exposing the population of PBMC to a CMV antigen; treating the exposed cells to isolated a subpopulation of cells enriched for activated cells expressing a TCR that binds to a CMV antigen; treating the subpopulation of cells to introduce a vector encoding a CAR that binds to an epitope on HIV gp120, thereby creating a population of bi-specific T cells expressing a TCR that binds to a CMV antigen and a CAR binds to an epitope on HIV gp120.

In various embodiments: the CAR comprises an antigen-binding domain that binds to an epitope on HIV gp120, a spacer, a transmembrane domain, a co-stimulatory domain, and a CD3 zeta signaling domain; the antigen-binding domain is a scFv comprising a heavy chain variable region and a light chain variable region; the heavy chain variable region has an amino acid sequence as shown in FIGS. 5A-5B, and wherein the light chain variable region has an amino acid sequence that is the corresponding light chain variable region sequence shown in FIGS. 5A-5B; the transmembrane domain is a CD4, CD8 or CD28 transmembrane domain; the co-stimulatory domain is a 4-1BB or CD28 co-stimulatory domain; the CAR comprises the amino acid sequence of any of SEQ ID NOS: 20-50 with up to 10 single amino acids substitutions; the CAR comprises the amino acid sequence of any of SEQ ID NOS: 20-50 with up to 5 single amino acids substitutions; the CAR comprises the amino acid sequence of any of SEQ ID NOS: 20-50 with up to 10 single amino acids substitutions; the T cells express a T cell receptor specific for a cytomegalovirus (CMV) antigen and are expanded in the presence of a T cell antigen before administration to the patient; the CMV antigen is a CMV peptide or a CMV vaccine; the expansion is in the presence of any HIV antigen and a CMV antigen; and the expansion is in the absence of an HIV antigen.

Described herein are CARs incorporating an scFv target to surface subunit of the HIV-1 envelope glycoprotein gp120 (e.g., one of the scFv described herein), which can be used alone or in various combinations to target HIV-infected cells. Among the gp120 binding sites that can be targeted are: V1/2 glycan, V1-2-3 glycan, V3 glycan, the mannose dependent site, CD4/V3, CD4 binding site, proximal CD4b spike, the membrane proximal region, gp41-120 interface. These binding sites and certain known antibodies targeting each are depicted in FIG. 3A and FIG. 3B. Thus, among the known scFv that can be incorporated into a CAR for use in the methods described herein are: 45-46, PGT121, PGDM1400, 3BC176, PGY126, PGT128, PGT145, PG9, PG16, VRC01, VRC07-523, X5, 35O22, 10E8, 3BNC117, and CD4-17b. For example, a combination of CAR constructs each incorporating one or more of N6, PGDM1400, PGT121 or 3BC176 scFv can be used. The pooled CAR vectors can be used to transduce the respective HIV subject-derived CMV-specific T cells that are then re-infused into the subject. Notably, the bi-specific nature of these CAR T cells allows CMV vaccine induced activation of the particular T cells.

### DESCRIPTION OF DRAWINGS

FIG. 1 is a schematic depiction of an example of a CMV-HIV CAR T cell. These CMV-specific T cells express: (1) an endogenous T cell receptor (TCR) complex that can be activated by MHC-presented CMV immunogenic peptides from the CMV-MVA Triplex vaccine; (2) chimeric antigen receptor (CAR) redirecting the T cells to gp120-expressing HIV-infected cells. The CAR construct contains the extracellular scFv domain of the broadly neutralizing antibody (bNAb) N6 with a linker, a transmembrane domain, a costimulatory domain (4-1BB), and the CD3ζ domain for T cell activation; and (3) a truncated human epidermal growth factor receptor (EGFRt) to serve as tracking/suicide element activated by cetuximab should CRS or neurotoxicity be observed.

FIG. 2 is a schematic depiction of a chimeric antigen receptor (CAR).

FIGS. 3A-3B are schematic depictions of neutralizing antibody sites on the gp120 HIV envelope protein. A shows various well documented antibody target epitopes with selected antibodies, including those used in swarm (PGDM1400, N6, PGT128, 3BC176). CD4+/CD8+ CAR T-cells are generated with the ability to target the PGDM1400, PGT121, N6 and 3BC176 binding sites. Each site can be targeted alone or in various combinations to avoid a priori the emergence of HIV viral mutations and resistance. B lists 16 different monoclonal antibodies that have been previously reported to potently suppress or neutralize HIV-infected cells by HIVgp120 targeting. An scFv from each has been cloned into a CAR. In each case the CAR uses the IgG4(L235E, N297Q) hinge, 4-1BB co-stimulatory domain and CD3 zeta stimulatory domain.

FIG. 4 is a schematic depiction of the CAR lentivirus construct. The construct contains an anti-gp120 scFv based on a broadly neutralizing antibody (bNAb), a spacer, a transmembrane domain, a 4-1BB costimulatory element a CD3 zeta domain, and a truncated human epidermal growth factor receptor (EGRFt).

FIGS. 5A-5B is a table providing examples of heavy chain and light chain antibody sequences that can be used to produce HIVgp120-targeted CAR.

FIG. 6 depicts FACS results showing the development of the gp120-8E5 cell line. Approximately 30% of the parental 8E5 cells express gp120 (left panel). Upon transduction with a lentiviral vector, transduced cells were sorted for eGFP and gp120 after 14 days in culture and 86.4% were double positive.

FIGS. 7A-7C depicts the results of a study showing the cytotoxic effect of various HIV-CAR T cells on gp120^{pos} 8E5-eGFP+ cells. Various CARs expressing the scFv of 45-46, N6, PGT121, PGT128, 3BC176 PDGM1400 and control 5S antibody (directed to HBV infection) were developed to target HIVgp120. In each case, the CAR construct includes the IgG4(L235E, N297Q) hinge, 4-1BB co-stimulatory domain and CD3 zeta stimulatory domain. The CAR T cells were then interrogated for killing at 1 to 1 (A), 1 to 2 (B), or 1 to 5 (C) ratio of CAR T cells to target gp120^{pos} 8E5-eGFP+ cells. A dose dependent effect is observed with the 45-46, N6, PGT128 and 3BC176-derived CARs, supporting the notion that these 4 CARs can be used as a swarm.

FIG. 8 depicts the results of an evaluation of the antiviral activity of HIV-CAR T cells on chronic HIV (NL4-3) infected Jurkat cells. CAR T cells targeting different binding sites on HIVgp120 were able to kill chronically HIV-infected Jurkat cells. CAR T cells targeting the EBV binding site S5 were used as negative control.

FIGS. 9A-9B depicts the results of an evaluation of the cytotoxicity of HIV-CAR T cells against gp120^{pos} 8E5-eGFP+ cells. HIV-CAR T cells expressing different anti-gp120 scFvs (WT, M1, and M2 45-46 bNAb and N6 bNAb as shown in Table 4) were generated from three healthy donors (HD459, HD460, and HD441). These HIV-CAR T cells were co-cultured with gp120^{pos} 8E5-eGFP+ cells at different E:T ratios. 96 hours later, cytotoxicity was determined by measuring GFP+ cells remaining in the co-culture. Cytotoxicity was normalized off of mock T-cell killing against gp120^{pos} 8E5-eGFP+ cells. B contains graphs showing antigen-driven proliferation of HIV-CAR T cells. To track T-cell proliferation, HIV-CAR T cells expressing different gp120-specific scFvs were stained with 2 μM Cell Trace Violet and co-cultured with varying concentrations of gp120^{pos} 8E5-eGFP+ cells (E:T ratios: 2:1, 1:1, and 0.5:1). 96 hours later, cells were harvested and stained with antibodies specific for T-cell markers and run on the flow cytometer. CAR T cells were gated by EGFR staining and proliferation was based off of CAR+ T cells. T cells in media served as negative control, while T-cells against LCL-OKT3 (a line engineered to express CD3 receptors) served as positive control.

FIGS. 10A-10B are a schematic depiction of HIV-CAR constructs with varying extracellular linker domains. Schematics of two HIV-CARs, each composed of a gp120-specific scFv, a linker domain (double mutation and Δ-CH2), and a costimulatory domain (41BB). The horizontal grey bars represent the plasma membrane. In A, the two linker domains shown are constructed as follows: double mutation contains a human IgG4 hinge region that has been mutated at two sites (red stripes represent L235E and N297Q mutations) to prevent binding by Fc. In B, Δ-CH2 was created from an IgG4 hinge region that was shortened by deleting the CH-2 domain.

FIGS. 11A-11C depicts the results of an evaluation of the cytotoxicity of HIV-CAR T-cells against gp120^{pos} 8E5-eGFP+ cells. HIV-CAR T-cells with different gp120-specific scFvs and either an EQ or dCH2 linker. The CAR constructs used are: A: WT, 45-46; B: M1 G54W; and C: M2 G54W, S28Y. CAR T-cells were co-cultured with gp120^{pos} 8E5-eGFP+ cells at different E:T ratios and 96 hours later, cytotoxic activity was determined by measuring GFP+ cells remaining in the co-culture. Cytotoxicity was normalized relative to mock T-cell killing of gp120^{pos} 8E5-eGFP+ cells.

FIG. 12 depicts the results of an evaluation of HIV-CAR T cells in an acute HIV NL4-3 infection. Jurkat cells were infected with HIV-1 NL4-3 (MOI=0.01) for 4 hours before co-culture with HIV-CAR T cells expressing the scFv of 45-46 bNAb targeting the CD4-binding site of HIV gp120, at 1:1, 1:2, 1:4, and 0:1 (control) ratios of CAR T cells to target HIV NL4-3-infected Jurkat cells. The HIV killing activities were evaluated by detecting p24 in ELISA assays on day 2, 4, 6, 8 and 12.

FIG. 13 depicts the result of an analysis of the antiviral activity of HIV-CAR T cells of chronically HIV-1 (NL4-3) infected Jurkat cells. 45-46 and N6-derived CAR T cells were able to kill chronically HIV-infected Jurkat cells at the 1:1 ratio and killing was normalized to that of non-CAR treated control infected Jurkat cells.

FIGS. 14A-14D depicts the results of HIV-CAR T cell killing of HIV infected cells. CD4+/CD8+ CAR T-cells were generated by transduction with a lentiviral vector containing an EGFR suicide/reporter gene and the broadly neutralizing 45-46 antibody targeted to the CD4 binding site in gp120. In A, various ratios of CAR T and J1.1. latently infected jurkat cells were co-cultured and the percent P24 inhibition was determined 10 days later. B shows a summary of the HIV-CAR T/J.1. PMA+ treated cell p24, % inhibition, and CAR vector expression as determined by EGFR expression. In C, various ratios of HIV-CAR T and Jurkat/NL4-3 infected cells were co-cultured and the neutralization of HIV-infected cells determined 10 days later. D shows a summary of the CAR T/Jurkat/NL4-3 treated cell p24, % inhibition, and CAR vector expression as determined by EGFR expression.

FIGS. 15A-15E depicts results from antiviral assay with CAR T [45-46 scFv] cells against five HIV-1 types. Killing of HIV-infected cells was measured at 10 days of co-culture. Each graph shows % of p24 inhibition (evaluated based on ELISA for p24 compared to the same cell treated with PBMC control) in presence of Jurkat cells infected with NL4-3 (T-tropic) (A); CEM cells infected with BaL (M-tropic) (B); 174× CEM cells infected with 89.6 (dual-tropic) (C), ACH-2, a latently HIV LAV infected CEM line (D); and J1.1, an HIV-1 LAV latently infected Jurkat cell line (E). HIV reactivation had been induced with PMA in latent ACH-2 and J1.1 cells.

FIGS. 16A-16D depicts immunohistochemistry results showing clearance of gp120-expressing cells from culture. HIVgp120/GFP positive 293T cells (green) were mixed with HIVgp120/Mcherry 293T cells (red) and then co-cultured alone (left panels A and C) or with HIV-specific CAR T cells (right panels) B and D). In the absence of CAR T cells, the mixture of cells is unchanged but in the presence of the CAR T cells, the HIV gp120/GFP positive 293T cells are eliminated. Compare day 0 [top panel] with day 4 [bottom panel]. These micrographs (10× magnification) are from a time-lapse video [4 images/h] that illustrates the cytotoxic effect of the HIV CAR T cells.

FIGS. 17A-17F contains schematic depiction (A), and FACS results (B-F), showing the development of CAR T cell swarms for targeted eradication of HIV. In A, two distinct methods are outlined for generating CD4+/CD8+ CAR T cell swarms. Approach (1) consists of co-transfecting two or greater vectors (n=2 are shown) into 293T producer cells, collecting the resultant supernatant for generating CAR T cells. Approach (2) consists of transfecting each CAR lentiviral vector separately and then pooling the resultant vectors together for direct addition to the impending CD4+/CD8+ CAR T cells. B shows negative FACS control for HT1080 cells. C shows single vector transduction with FACS for GFP. D shows single vector transduction with FACS for mCherry. E shows FACS of vectors generated by approach (1)—the single production swarm resulting from a co-transfection of both the GFP and mCherry expressing lentiviral vectors. F shows FACS of vector transduced cells using vectors generated by approach (2), dual production swarm.

FIG. 18 is a bar graph showing the results of a study showing that combinations of two or three distinct HIV-CAR T cells are more effective than single HIV-CAR T cells against a resistant HIV strain YU2-N332S (resistant to PGT128).

FIGS. 19A-19B depicts the results of a study of CMV and HIV-specific reactivity of CMV-HIV CAR T cells. (A) To demonstrate specific antigen stimulated proliferative capacity, CMV-HIV CAR T cells or CMV-T cells were stained with 5 uM of fluorescent CellTrace Violet dye. Stained T cells were then co-cultured with at a 1:1 E:T ratio. Eight days later, cells were harvested and stained for surface markers and acquired on a MACSQuant flow cytometer. Dilution of CellTrace Violet dye indicated cell proliferation only in presence of LCL-OKT3 and CMVpp65+ pulsed cells, and not in presence of negative controls. (B) CMV-HIV CAR T cells or CMV T cells were co-cultured with GFP+ lines: KG-1A (negative control) and 8E5 (gp120pos target line) at several E:T ratios (2:1, 1:1, 1:5). Percent of cytotoxicity was determined by measuring GFP+ cells remaining in the culture and normalized off of GFP+ cells remaining from autologous PBMCs cultured with respective target lines.

FIGS. 20A-20B depicts the results of studies characterizing CMV-HIV CAR T cells generated using PBMC from HIV infected donors. (A) Purity of CMV-reactive cells isolated by IFNγ+ selection on a CliniMACS Prodigy. (B) The following day, cells were transduced with a LV encoding HIV-directed CAR at a MOI of 3. The presence of CAR T cells was measured by staining with an antibody against EGFR (Erbitux).

FIGS. 21A-21C depict the results of a phenotypic characterization of CMV-HIV CAR T cells from HIV-infected donor. (A) Percent of CD4+ and CD8+ T cells in HIV-CAR T cells. The cell product was gated on EGFR+ cells and analyzed for the central memory/stem marker CD62L (B) and early stage memory cell marker and co-stimulatory molecule CD27 (TCM and Naïve cells) as well as CD45RA (TEMRA) (C).

FIG. 22 depicts the results of an analysis of exhaustion markers in CMV-HIV CAR T cells prepared from an HIV-infected donor. CMV-HIV CAR T cells were derived from an HIV-infected subject on ART and propagated in the presence of IL-2 (50 U/mL) and IL15 (1 ng/mL) for 22 days in T cell medium (10% FBS in RPMI). Expression of exhaustion markers such as PD1, LAG3 and Tim3 was analyzed by flow cytometry. Percentages of positive cells are depicted.

FIG. 23 depicts the results of a study of CMV reactivity of the CMV-HIV CAR T cells prepared from an HIV-infected donor. To determine if CMV-HIV CAR T cells could still respond to CMV, autologous PBMCs were loaded with CMVpp65 peptides for 2 hours to be used as antigen presenting cells (APC). CMV-HIV CAR T cells were then co-cultured with these APCs for 24 hours and stained for intracellular IFNγ to determine a CMV-specific response.

FIGS. 24A-24C depicts the results of a study of CMV-HIV CAR T cells prepared from HIV-infected donors. CMV-HIV CAR T cells or CMV-CD19 CAR T cells (Ctr) were manufactured from an HIV-infected donor. (A) Levels of p24 were measured in the supernatant by ELISA on day 20 and normalized to the level in the control condition. (B) CAR T cells were co-cultured at different E:T ratios with HIV NL4-3 infected Jurkat-GFP+ cells. The cytotoxicity of the CAR T cells was evaluated on day 7 by measuring the percent of killed GFP+ cells. The level of p24 was measured in the supernatants by ELISA on day 20 (C).

FIG. 25 is a schematic depiction of the generation of hu-PBL-NSG mouse model of HIV and CMV-HIV CAR T cells. PBMCs were collected from a donor and CMV-reactive T cells were then enriched as described to manufacture CMV-HIV (N6) CAR T cells. CS1 (also known as CD319) is a tumor associated antigen that is only expressed on multiple myeloma cells and was chosen to design the control CAR T cells. The CMV negative fraction of autologous PBMCs was cryopreserved and fractions were thawed prior to use. 2×106 CMV-negative PBMCs were pre-challenged with HIV-1Bal for two days prior to IP injection, and at the same time, 8×106 CMV-negative PBMCs (that had not been challenged with HIV) were injected. Mice that exhibited engraftment (defined as >5% huCD45+ cells in peripheral blood) and HIV infection (defined as >103 gc/mL of HIV in blood) were selected for immunotherapy on Day 14. Mice received a vaccination with CMV-peptide coated autologous CMV negative PBMCs on Day 32 and mice were sacrificed on Day 55.

FIG. 26 depicts the results of a study of the expansion of CMV-HIV CAR T cells from HIV-infected subject in a hu-PBL-NSG mouse model of HIV. Blood (50-100 μL) was collected from retro orbital vein at two-week intervals after transplantation of human PBMCs and HIV-1 infection. Cells from blood were stained with antibodies against huCD45, huCD3, huCD4, and huCD8 and analyzed for cellular composition by FACS (not shown). Additionally, cells were stained by surface expression of huEGFR, which is expressed as a transgenic marker in the bicistronic lentiviral vector in CMV-HIV CAR T cells. Data for individual mice (n=9) are shown, with lines marking mean values and standard errors of means (SEM). Statistical analysis was performed by two-tailed, paired t-test (*, p<0.05; **, p<0.005).

FIGS. 27A-27D depicts the results of detection of CMV-HIV CAR T cells in the bone marrow and peripheral blood of hu-PBL-NSG mouse model of HIV. Flow cytometric analyses of EGFR+ CAR T cells from HIV-infected subject 6 weeks post CAR T cell infusion in mice. (A) Relative distribution of CAR T cells in tissues, in individual mice. (B) Frequency of CD4+ and CD8+ T cells within the CAR T cell fraction. (C, D) Frequency of cells expressing CD62L or CD27 within the CD4+ and CD8+ T cell population.

FIGS. 28A-28C depicts the results of a study showing that CMV-HIV CAR T cells expand in vivo upon CMV vaccination and support expansion of CD4+ T cells during HIV infection. Percentages of EGFR+ CAR T cells (A) or CD4+ T cells (B) were analyzed by FACS one day prior (Day 31) or six days after CMV vaccination (Day 38). Statistical analysis was performed by two-tailed, unpaired t-test when comparing different groups or a two-tailed, paired t-test when analyzing individual mice over time (*, p<0.05; **, p<0.01; ***, p<0.001). (C) Blood was collected to analyze levels of HIV RNA in serum over the 55-day experiment. Data points represent mean values (n=5) and above error bars represent standard error of mean (SEM).

FIG. 29 is a schematic depiction of the generation of Hu-PBL-NSG mice and HIV-CAR T cell infusion study design.

FIG. 30 is a graph showing that HIV-CAR T cells increase survival of humanized PBL-SCID mouse model under HIV-1 infection.

FIG. 31 is a schematic flow chart showing proof-of-concept study design.

FIGS. 32A-43B contain textual graphs and tables showing the amino acid sequences of various HIV-CARs and the various domains of these CARs.

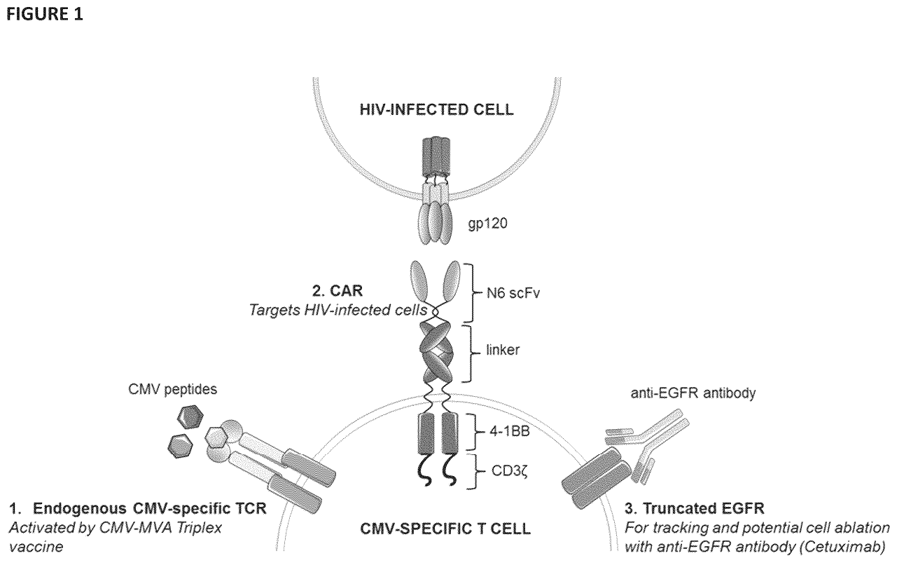

### DETAILED DESCRIPTION

### HIV-Targeted CAR

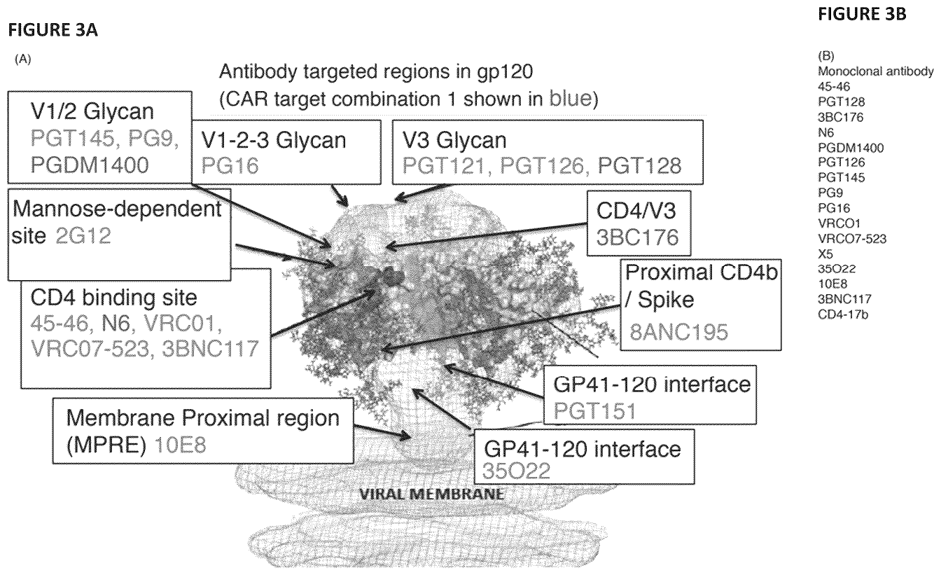

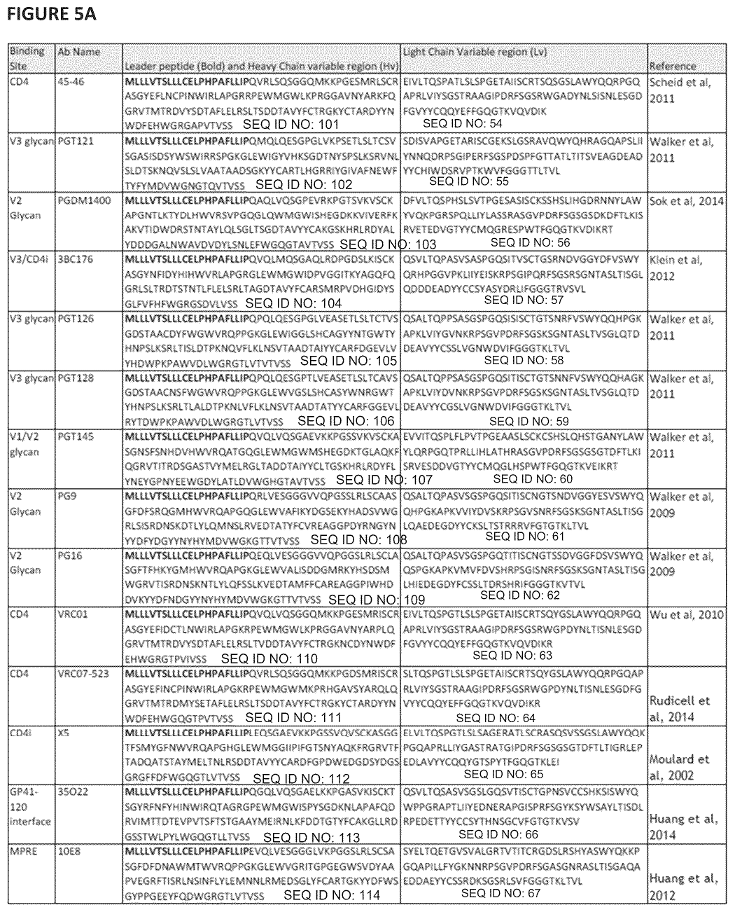

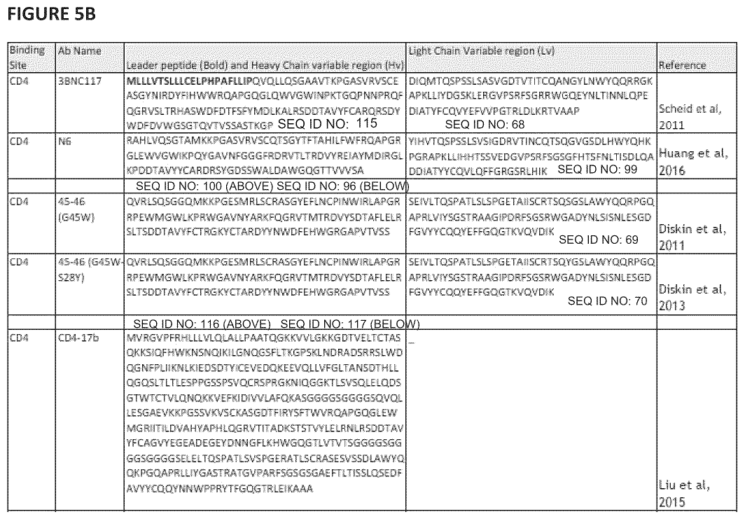

The HIV-targeted CARs described herein include a HIVgp120-targeting scFv (e.g., a scFv including a heavy chain and light chain pair from those in FIGS. 3A-3B or FIGS. 5A-5B).

Useful HIV-CAR consist of or comprises the amino acid sequence of any of SEQ ID NOs:29-40 (mature CAR lacking a signal sequence) or the HIV CAR consists of or comprises the amino acid sequence of any of SEQ ID NOs: 41-53 (immature CAR having a GMCSFRa signal sequence). The CAR and can be expressed in a form that includes a signal sequence, e.g., a human GM-CSF receptor alpha signal sequence (MLLLVTSLLLCELPHPAFLLIP; SEQ ID NO:26). The CAR can be expressed with additional sequences that are useful for monitoring expression, for example, a T2A skip sequence and a truncated EGFRt. Thus, the CAR can comprise or consist of the amino acid sequence of any of SEQ ID Nos: 29-40 or can comprise or consist of an amino acid sequence that is at least 95%, 96%, 97%, 98% or 99% identical to any of SEQ ID Nos: 29-40. The CAR can comprise or consist of the amino acid sequence of any of SEQ ID Nos: 29-40 with up to 1, 2, 3, 4 or 5 amino acid changes (preferably conservative amino acid changes).

### Spacer Region

The CAR described herein can include a spacer located between the HIV targeting domain (i.e., a gp120-targeted ScFv or variant thereof) and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG (SEQ ID NO: 2) |
| IgG4 hinge) | 12 aa | ESKYGPPCPPCP (SEQ ID NO: 3) |
| (S→P | | |
| (S228P) | | |
| IgG4 hinge | 12 aa | ESKYGPPCPSCP (SEQ ID NO: 4) |
| IgG4 hinge | 22 aa | ESKYGPPCPPCPGGGSSGGGSG |
| (S228P) + | | (SEQ ID NO: 5) |
| linker | | |
| CD28 hinge | 39 aa | IEVMYPPPYLDNEKSNGTIIHVKGKHL |
| | | CPSPLFPGPSKP (SEQ ID NO: 6) |
| CD8 hinge- | 48 aa | AKPTTTPAPRPPTPAPTIASQPLSL |
| 48 aa | | RPEACRPAAGGAVHTRGLDFACD |
| | | (SEQ ID NO:7) |
| CD8 hinge- | 45 aa | TTTPAPRPPTPAPTIASQPLSLRPE |
| 45 aa | | ACRPAAGGAVHTRGLDFACD |
| | | (SEQ ID NO: 8) |
| IgG4(HL-CH3) | 129 aa | ESKYGPPCPPCPGGGSSGGGSGGQPR |
| (includes | | EPQVYTLPPSQEEMTKNQVSLTCLVK |
| S228P | | GFYPSDIAVEWESNGQPENNYKTTPP |
| in hinge) | | VLDSDGSFFLYSRLTVDKSRWQEGNV |
| | | FSCSVMHEALHNHYTQKSLSLSLGK |
| | | (SEQ ID NO: 9) |
| IgG4(L235E, | 229 aa | ESKYGPPCPSCPAPEFEGGPSVFLFPP |
| N297Q) | | KPKDTLMISRTPEVTCVVVDVSQEDPE |
| | | VQFNWYVDGVEVHQAKTKPREEQFQ |
| | | STYRVVSVLTVLHQDWLNGKEYKCK |
| | | VSNKGLPSSIEKTISKAKGQPREPQVY |
| | | TLPPSQEEMTKNQVSLTCLVKGFYPS |
| | | DIAVEWESNGQPENNYKTTPPVLDSD |
| | | GSFFLYSRLTVDKSRWQEGNVFSCSV |
| | | MHEALHNHYTQKSLSLSLGK |
| | | (SEQ ID NO: 10) |
| IgG4(S228P, | 229 aa | ESKYGPPCPPCPAPEFEGGPSVFLFPP |
| L235E, N297Q) | | KPKDTLMISRTPEVTCVVVDVSQEDPE |
| | | VQFNWYVDGVEVHQAKTKPREEQFQ |
| | | STYRVVSVLTVLHQDWLNGKEYKCK |
| | | VSNKGLPSSIEKTISKAKGQPREPQVY |
| | | TLPPSQEEMTKNQVSLTCLVKGFYPS |
| | | DIAVEWESNGQPENNYKTTPPVLDSD |
| | | GSFFLYSRLTVDKSRWQEGNVFSCSV |
| | | MHEALHNHYTQKSLSLSLGK |
| | | (SEQ ID NO: 11) |
| IgG4(CH3) | 107 aa | GQPREPQVYTLPPSQEEMTKNQVSLT |
| | | CLVKGFYP SDIAVEWESNGQPENNYK |
| | | TTPPVLDSDGSFFLYSRLTVDKSRWQ |
| | | EGNVFSCSVMHEALHNHYTQKSLSLS |
| | | LGK (SEQ ID NO: 12) |

Some spacer regions include all or part of an immunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain or both a CH3 domain and a CH2 domain. The immunoglobulin derived sequences can include one ore more amino acid modifications, for example, 1, 2, 3, 4 or 5 substitutions, e.g., substitutions that reduce off-target binding.

The hinge/linker region can also comprise a IgG4 hinge region having the sequence ESKYGPPCPSCP (SEQ ID NO:4) or ESKYGPPCPPCP (SEQ ID NO:3).

The hinge/linger region can also comprise the sequence ESKYGPPCPPCP (SEQ ID NO:3) followed by the linker sequence GGGSSGGGSG (SEQ ID NO:2) followed by IgG4 CH3 sequence GQPREPQVYTLPPSQEEMTKNQVSLTCLVKGFYPSDIA VEWESNGQPENNYK TTPPVLDSDGSFFLYSRLTVDKSRWQEGNVFSCSVMHEALHNHYTQKSLSLS LGK (SEQ ID NO:12). Thus, the entire linker/spacer region can comprise the sequence: ESKYGPPCPPCPGGGSSGGGSGGQPREPQVYTLPPSQEEMTKNQVSLTCLVK GFYPSDIAVEWESNGQPENNYKTTPPVLDSDGSFFLYSRLTVDKSRWQEGNV FSCSVMHEALHNHYTQKSLSLSLGK (SEQ ID NO:9). In some cases, the spacer has 1,2,3,4, or 5 single amino acid changes (e.g., conservative changes) compared to SEQ ID NO:9. In some cases, the IgG4 Fc hinge/linker region that is mutated at two positions (L235E; N297Q) in a manner that reduces binding by Fc receptors (FcRs).

### Transmembrane Domain

A variety of transmembrane domains can be used in the. Table 2 includes examples of suitable transmembrane domains. Where a spacer region is present, the transmembrane domain is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL |
| | | | (SEQ ID NO: 13) |
| CD28 | NM_006139 | 27 aa | FWVLVVVGGVLACYSLLVTVA |
| | | | FIIFWV |
| | | | (SEQ ID NO: 14) |
| CD28(M) | NM_006139 | 28 aa | MFWVLVVVGGVLACYSLLVTV |
| | | | AFIIFWV |
| | | | (SEQ ID NO: 15) |
| CD4 | M35160 | 22 aa | MALIVLGGVAGLLLFIGLG |
| | | | IFF |
| | | | (SEQ ID NO: 16) |
| CD8tm | NM_001768 | 21 aa | IYIWAPLAGTCGVLLLSLVIT |
| | | | (SEQ ID NO: 17) |
| CD8tm2 | NM_001768 | 23 aa | IYIWAPLAGTCGVLLLSLVI |
| | | | TLY |
| | | | (SEQ ID NO: 18) |
| CD8tm3 | NM_001768 | 24 aa | IYIWAPLAGTCGVLLLSLVI |
| | | | TLYC |
| | | | (SEQ ID NO: 19) |
| 41BB | NM_001561 | 27 aa | IISFFLALTSTALLFLLFFLT |
| | | | LRFSVV |
| | | | (SEQ ID NO: 20) |

### Costimulatory Domain

The costimulatory domain can be any domain that is suitable for use with a CD3ζ signaling domain. In some cases, the costimulatory domain is a CD28 costimulatory domain that includes a sequence that is at least 90%, at least 95%, at least 98% identical to or identical to: RSKRSRGGHSDYMNMTPRRPGPTRKHYQPYAPPRDFAAYRS (SEQ ID NO:23; LL to GG amino acid change double underlined). In some cases, the CD28 co-signaling domain has 1, 2, 3, 4 of 5 amino acid changes (preferably conservative and preferably not in the underlined GG sequence) compared to SEQ ID NO:23. In some cases the co-signaling domain is a 4-1BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:24). In some cases, the 4-1BB co-signaling domain has 1, 2, 3, 4 of 5 amino acid changes (preferably conservative) compared to SEQ ID NO:24.

The costimulatory domain(s) are located between the transmembrane domain and the CD3ζ signaling domain. Table 3 includes examples of suitable costimulatory domains together with the sequence of the CD3ζ signaling domain.

TABLE 3
CD3ζ Domain and Examples
of Costimulatory Domains

| Name | Accession | Length | Sequence |
|---|---|---|---|
| CDζ | J04132.1 | 113 aa | RVKFSRSADAPAYQQGQNQL |
| | | | YNELNLGRREEYDVLDKRRG |
| | | | RDPEMGGKPRRKNPQEGLYN |
| | | | ELQKDKMAEAYSEIGMKGER |
| | | | RRGKGHDGLYQGLSTATKDT |
| | | | YDALHMQALPPR |
| | | | (SEQ ID NO: 21) |
| CD28 | NM_006139 | 42 aa | RSKRSRLLHSDYMNMTPRRP |
| | | | GPTRKHYQPYAPPRDFAAYR |
| | | | S |
| | | | (SEQ ID NO: 22) |
| CD28gg* | NM_006139 | 42 aa | RSKRSRGGHSDYMNMTPRRP |
| | | | GPTRKHYQPYAPPRDFAAYR |
| | | | S |
| | | | (SEQ ID NO: 23) |
| 41BB | NM_001561 | 42 aa | KRGRKKLLYIFKQPFMRPVQ |
| | | | TTQEEDGCSCRFPEEEEGGC |
| | | | EL |
| | | | (SEQ ID NO: 24) |
| OX40 | | 42 aa | ALYLLRRDQRLPPDAHKPPG |
| | | | GGSFRTPIQEEQADAHSTLA |
| | | | KI |
| | | | (SEQ ID NO: 25) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and in some cases a short linker consisting of 2-10, e.g., 3 amino acids (e.g., GGG) is positioned between the costimulatory domain and the CD3ζ signaling domain.

### CD3ζ Signaling Domain

The CD3ζ Signaling domain can be any domain that is suitable for use with a CD3ζ signaling domain. In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPR RKNPQEGLYNELQKDKMAEAYSEIGMKGERRRGKGHDGLYQGLSTATKDT YDALHMQALPPR (SEQ ID NO:21). In some cases, the CD3ζ signaling has 1, 2, 3, 4 of 5 amino acid changes (preferably conservative) compared to SEQ ID NO:21.

### Truncated EGFR

The CD3ζ signaling domain can be followed by a ribosomal skip sequence (e.g., LEGGGEGRGSLLTCGDVEENPGPR; SEQ ID NO:27) and a truncated EGFR having a sequence that is at least 90%, at least 95%, at least 98% identical to or identical to: LVTSLLLCELPHPAFLLIPRKVCNGIGIGEFKDSLSINATNIKHFKNCTSISGDLH ILPVAFRGDSFTHTPPLDPQELDILKTVKEITGFLLIQAWPENRTDLHAFENLEII RGRTKQHGQFSLA VVSLNITSLGLRSLKEISDGDVIISGNKNLCYANTINWKK LFGTSGQKTKIISNRGENSCKATGQVCHALCSPEGCWGPEPRDCVSCRNVSR GRECVDKCNLLEGEPREFVENSECIQCHPECLPQAMNITCTGRGPDNCIQCAH YIDGPHCVKTCPAGVMGENNTLVWKYADAGHVCHLCHPNCTYGCTGPGLE GCPTNGPKIPSIATGMVGALLLLLVVALGIGLFM (SEQ ID NO:28). In some cases, the truncated EGFR has 1, 2, 3, 4 of 5 amino acid changes (preferably conservative) compared to SEQ ID NO:28.

### HIV-Targeted CAR

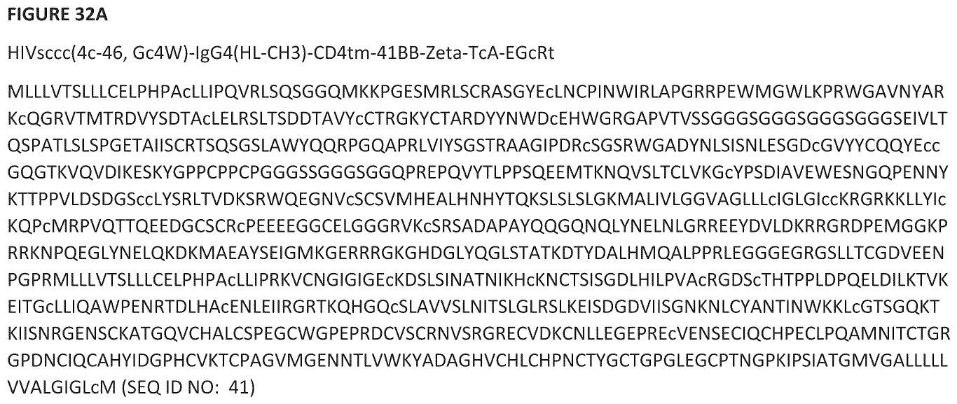

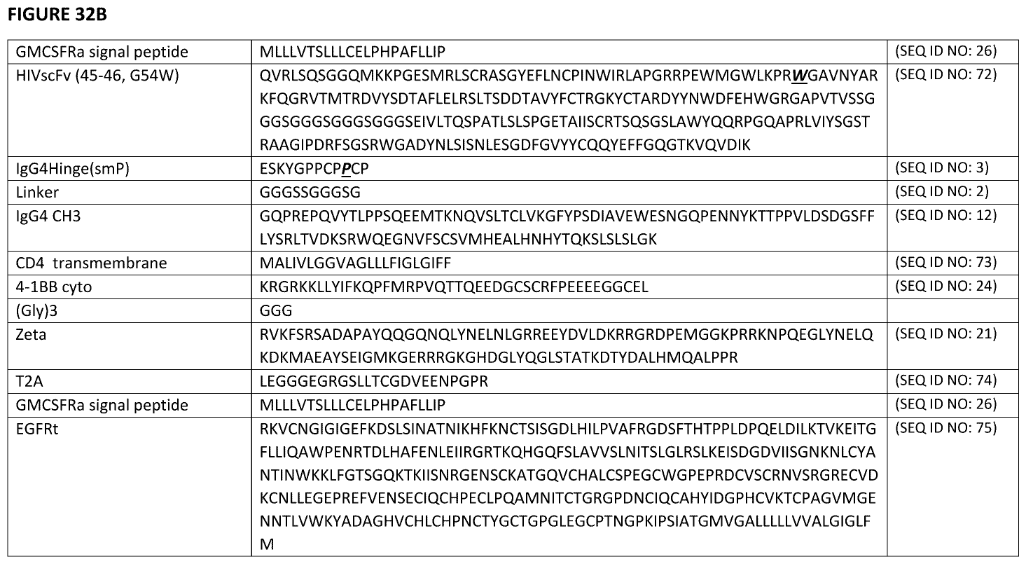

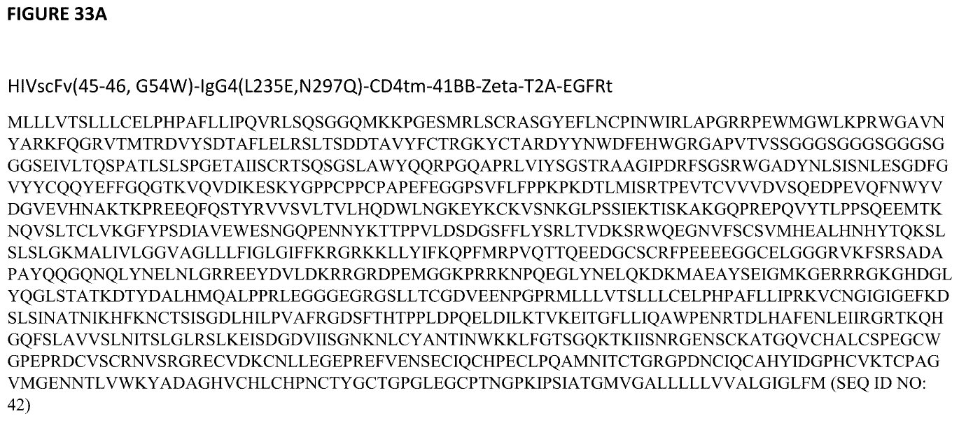

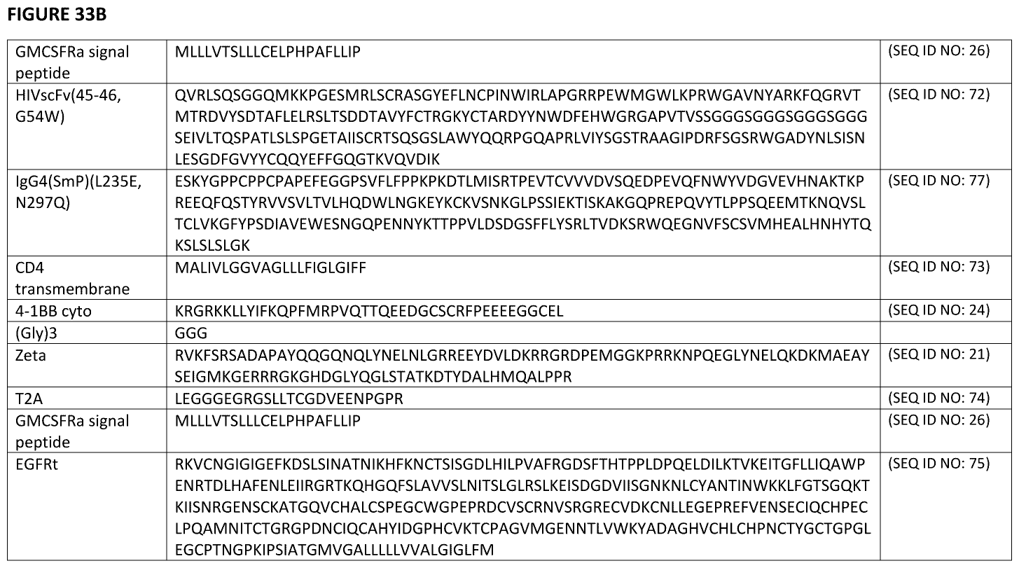

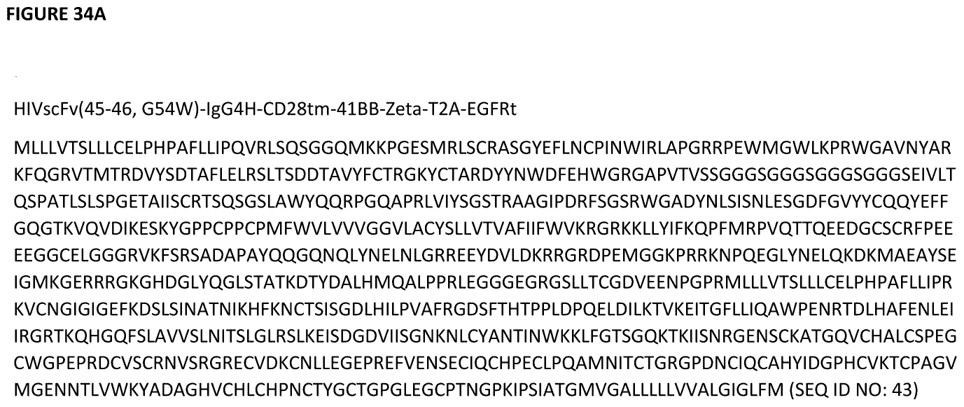

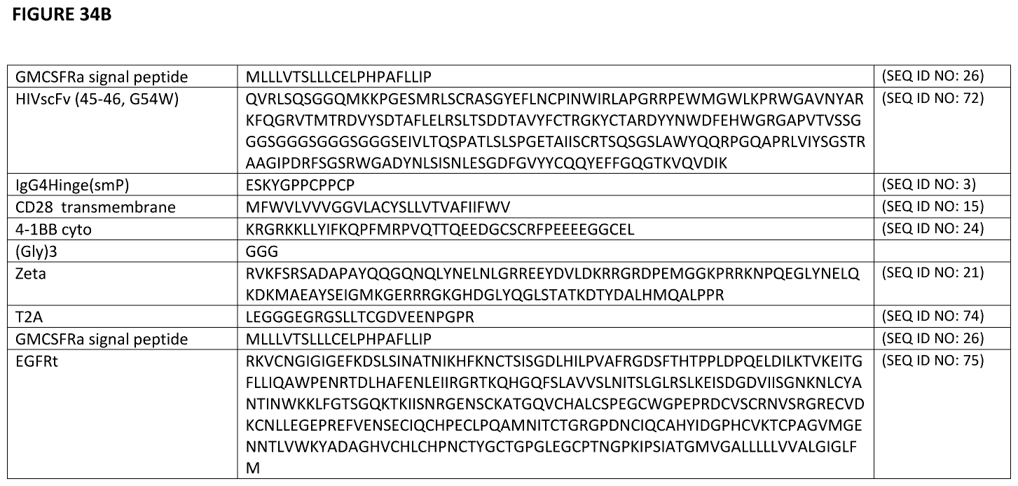

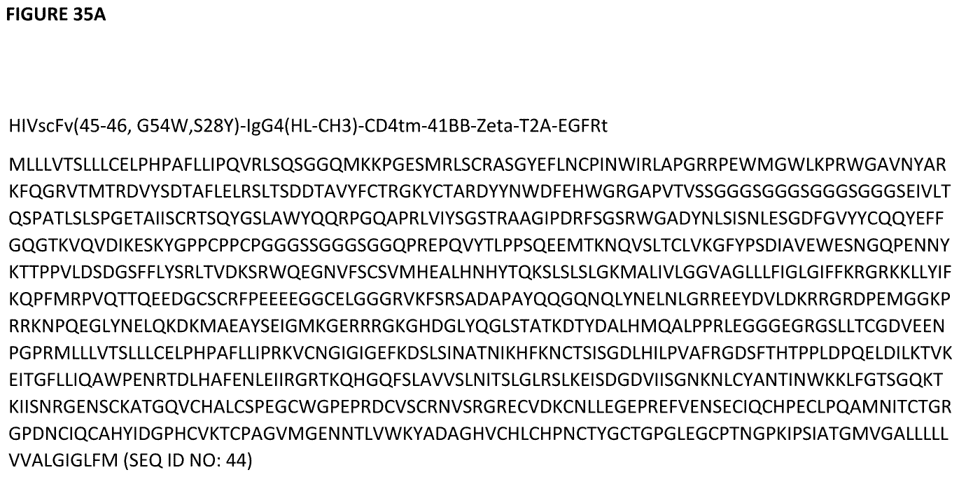

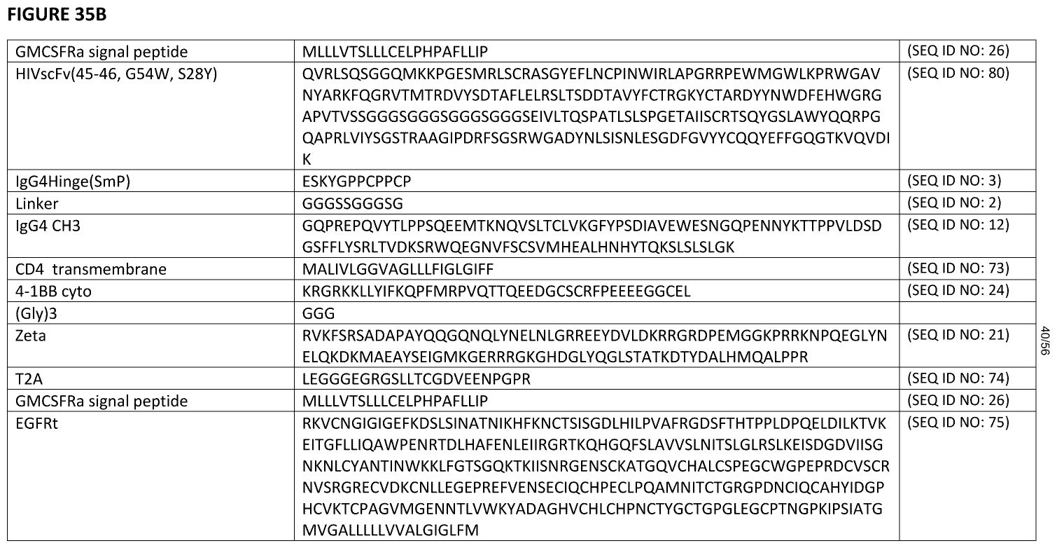

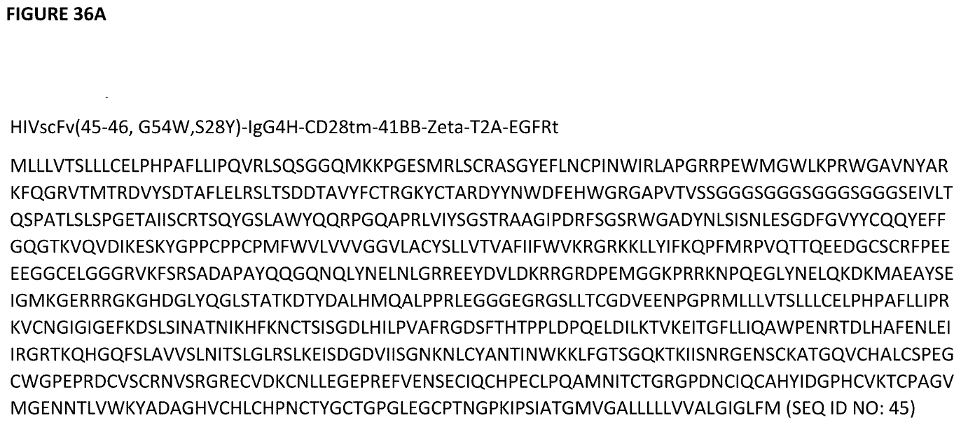

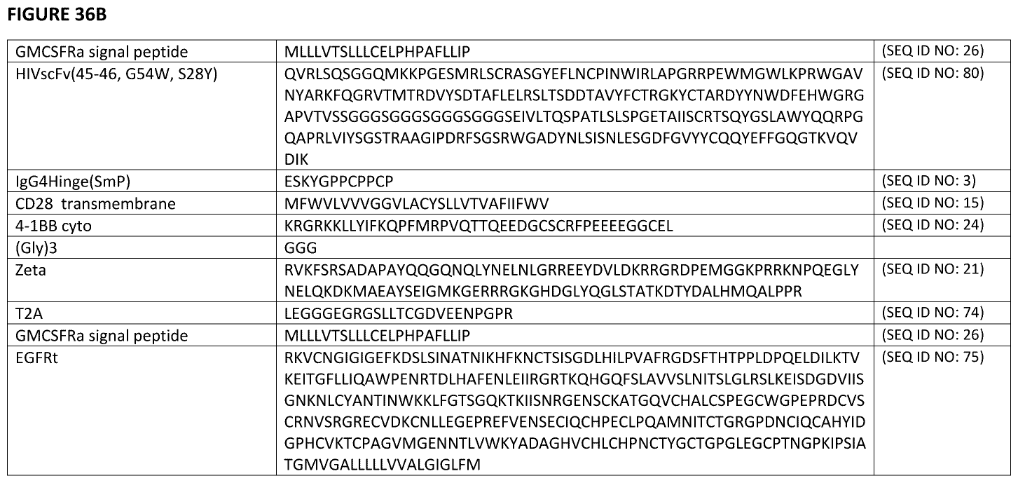

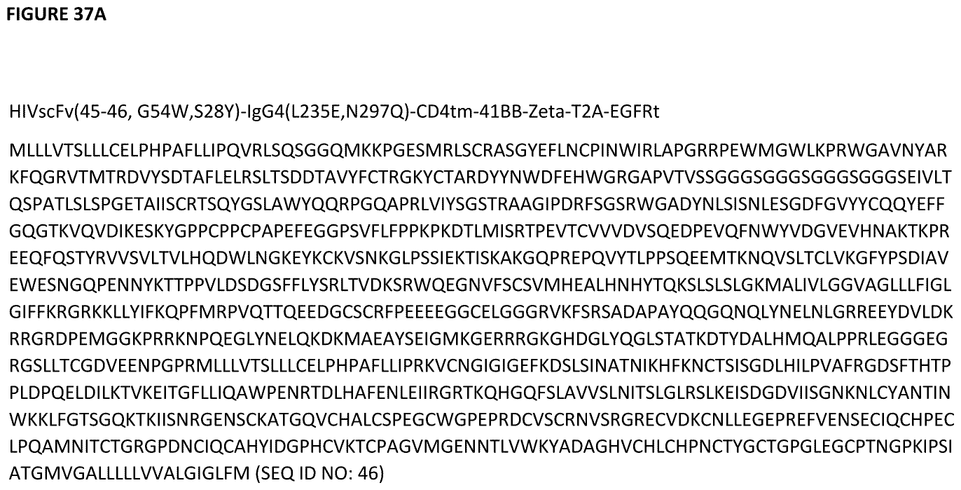

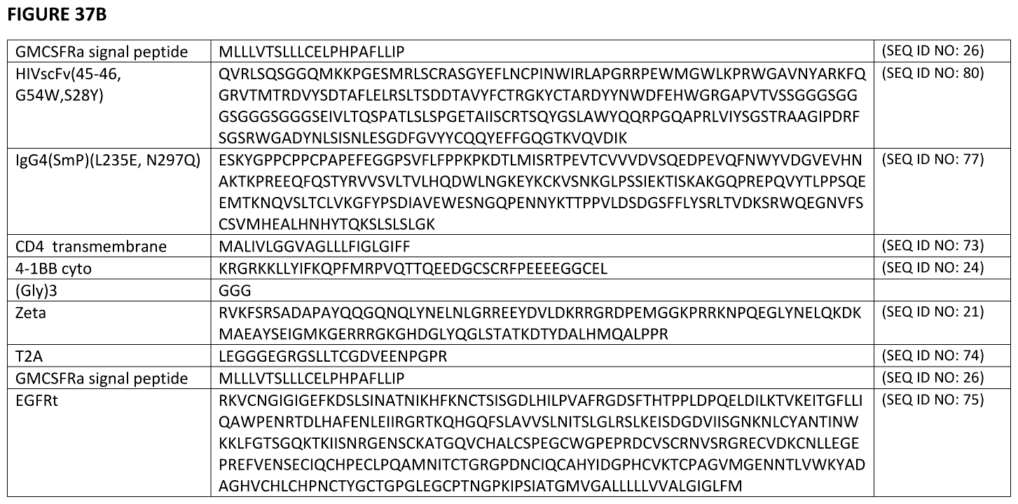

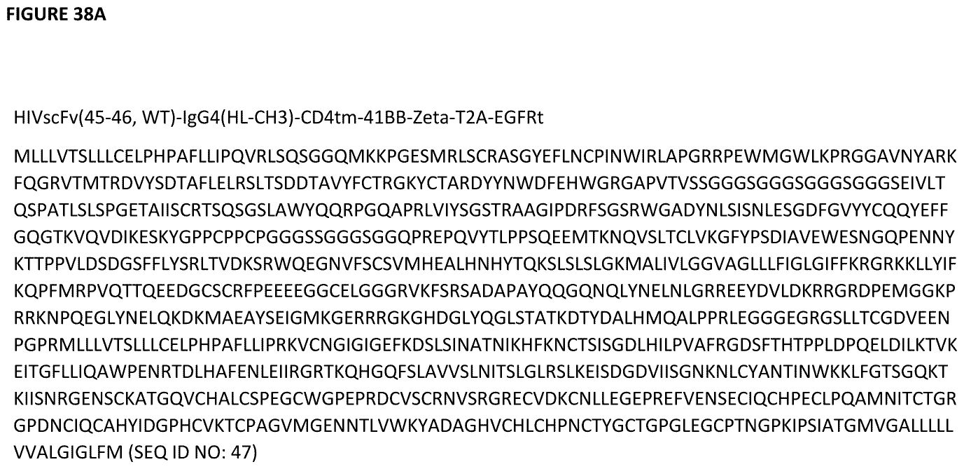

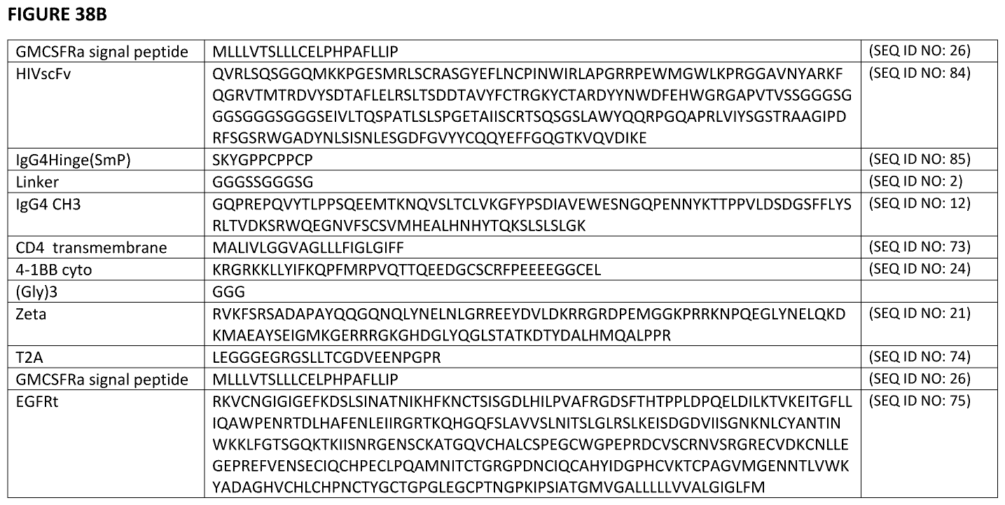

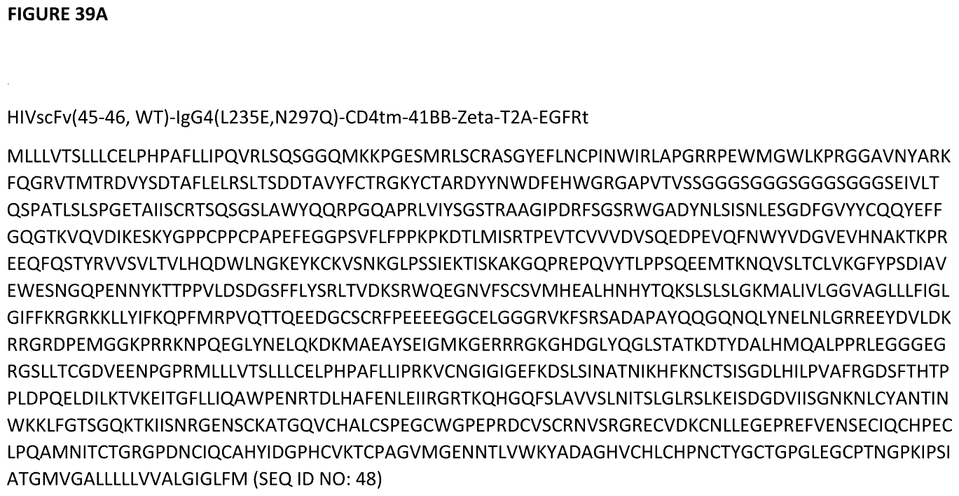

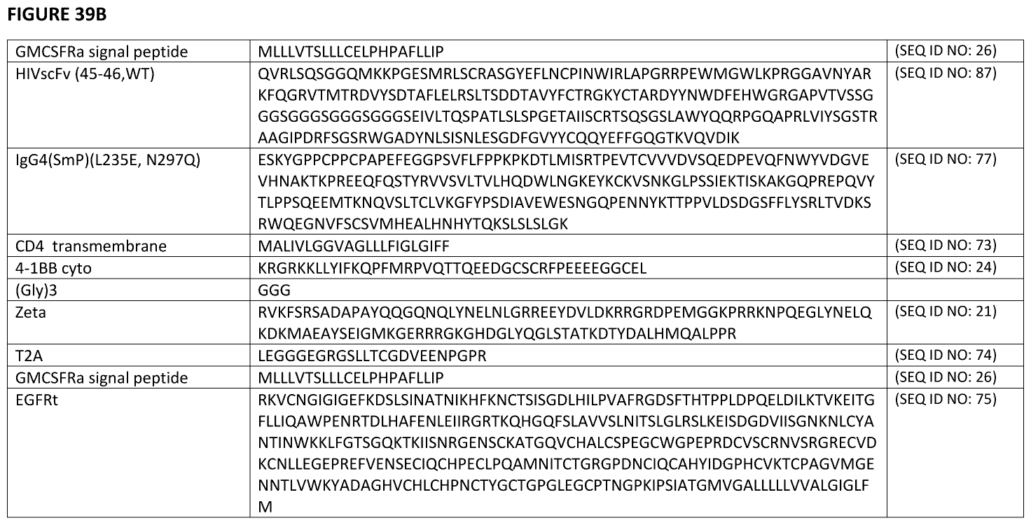

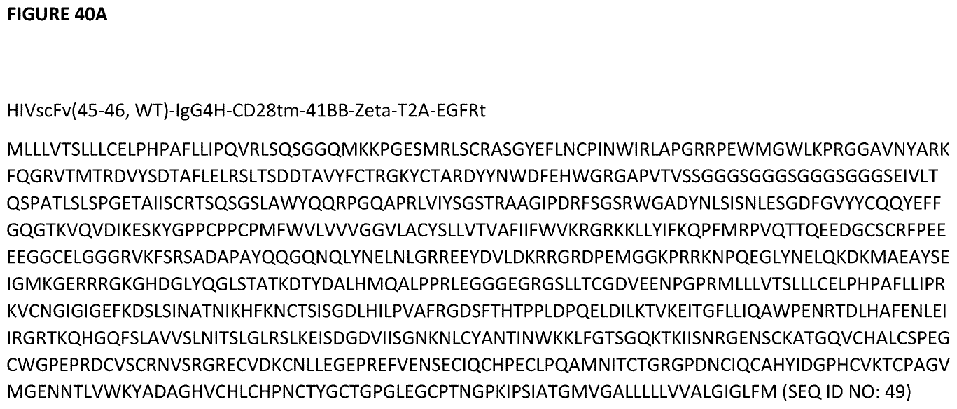

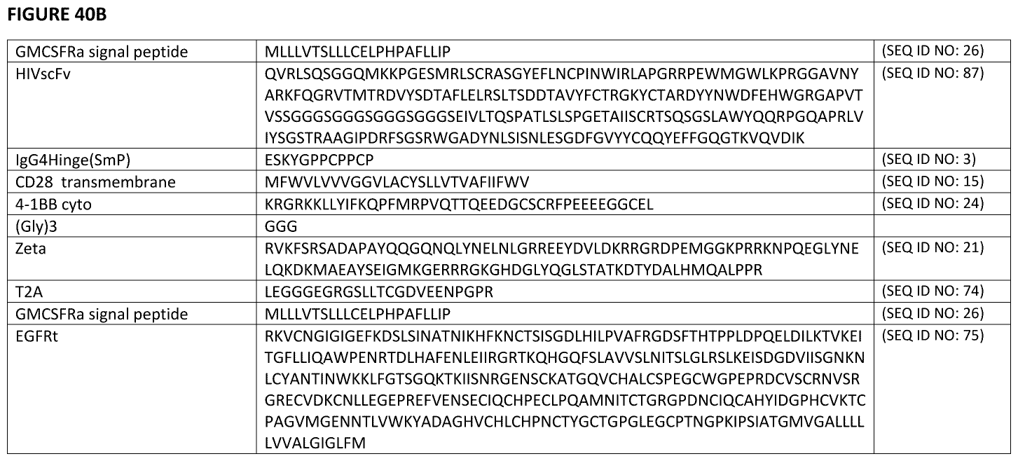

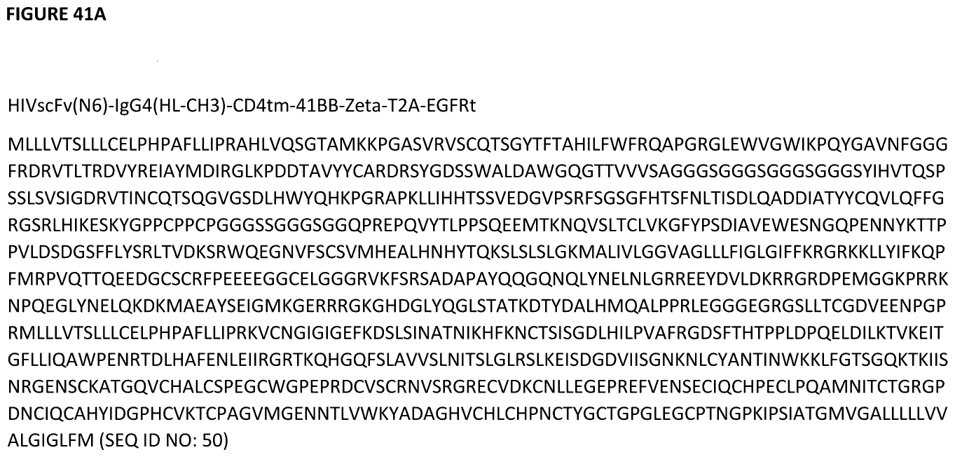

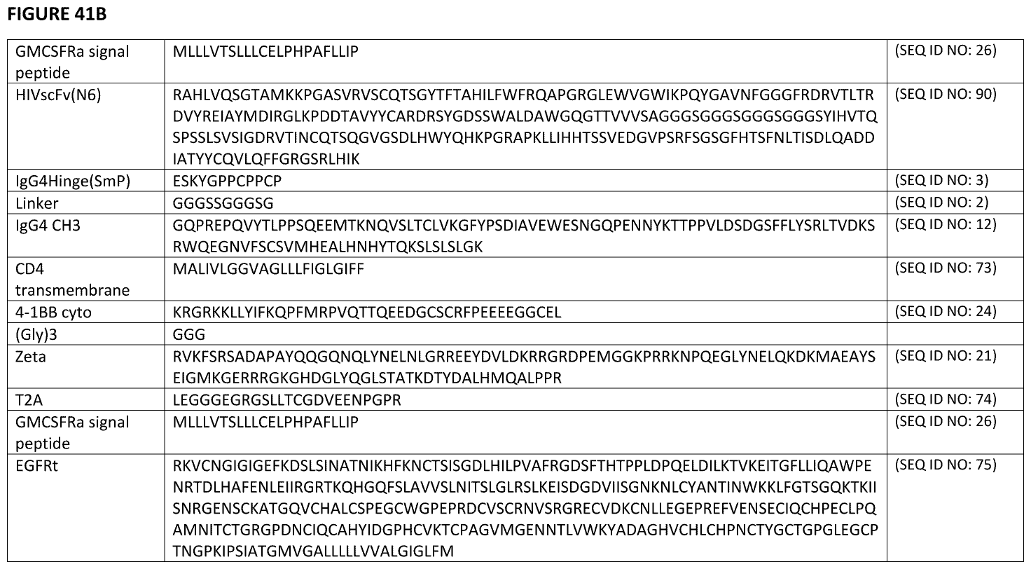

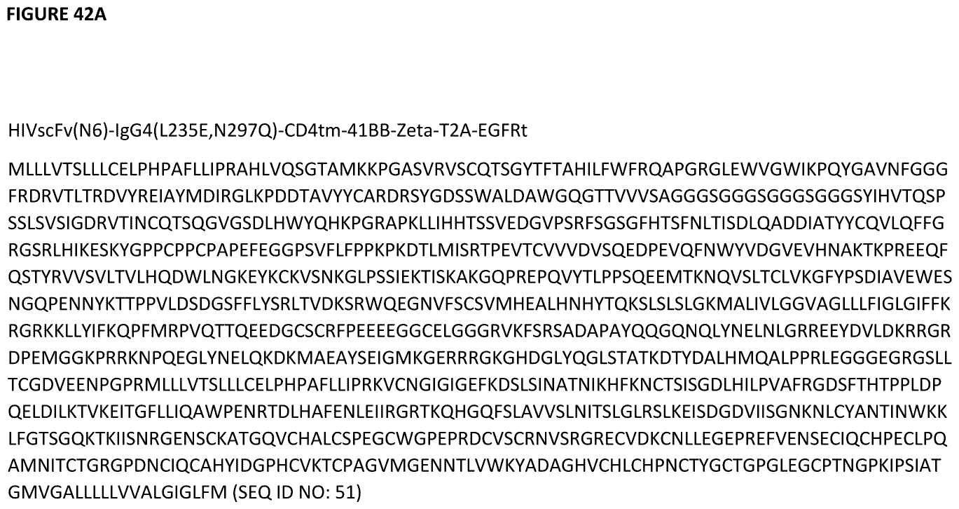

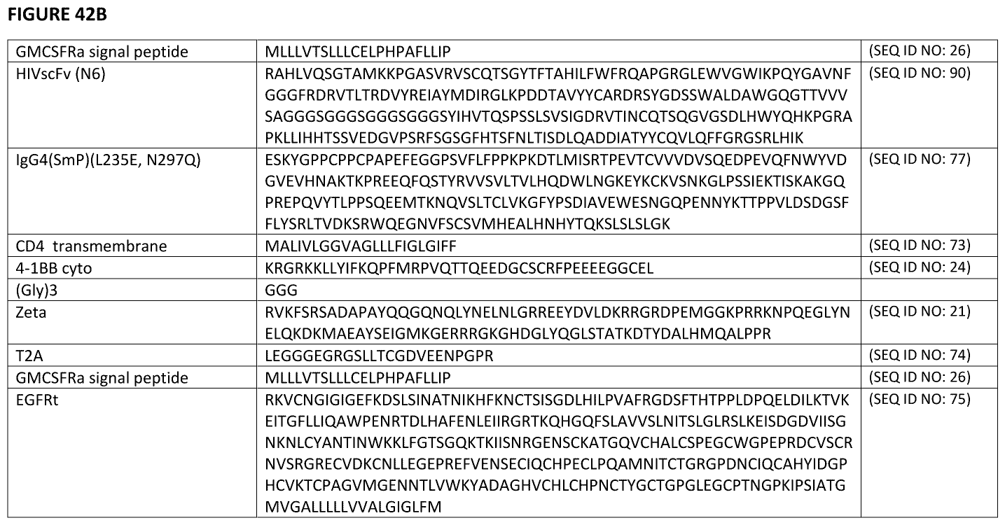

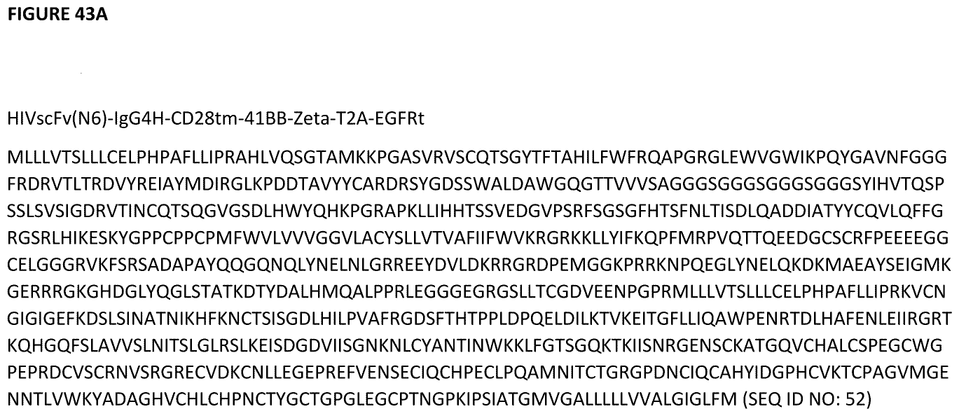

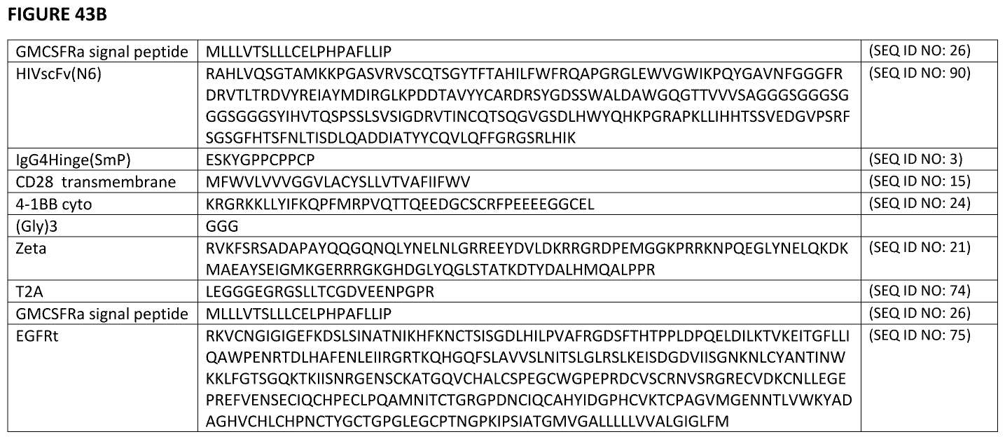

FIGS. 32-43 have the sequence of various HIV-targeted CAR. In each case the full amino acid sequence includes a GMSCFRa signal sequence and an EGFRt sequence that are not present in the mature CAR as expressed on T cells. The mature CAR sequences (lacking GMSCFRa and EGFRt) in FIGS. 32-43 are SEQ ID NOS: 29-40, respectively. An individual infected by HIV can be administered a population of human T cells transduced by a vector comprising an expression cassette encoding at least one (e.g, 1, 2, 3, 4, 5 or 6) HIV chimeric antigen receptor described herein (e.g., a CAR that comprises or consists of the amino acid sequence of any of SEQ ID Nos: 29-40 or an amino acid sequence that is at least 95%, 96%, 97%, 98% or 99% identical to any of SEQ ID Nos: 29-40 or the amino acid sequence of any of SEQ ID Nos: 29-40 with up to 1, 2, 3, 4 or 5 amino acid changes (preferably conservative amino acid changes). In various embodiments: the population of human T cells comprise CD4+ and/or CD8+ cells.

An amino acid modification refers to an amino acid substitution, insertion, and/or deletion in a protein or peptide sequence. An “amino acid substitution” or “substitution” refers to replacement of an amino acid at a particular position in a parent peptide or protein sequence with another amino acid. A substitution can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. The following are examples of various groupings of amino acids: 1) Amino acids with nonpolar R groups: Alanine, Valine, Leucine, Isoleucine, Proline, Phenylalanine, Tryptophan, Methionine; 2) Amino acids with uncharged polar R groups: Glycine, Serine, Threonine, Cysteine, Tyrosine, Asparagine, Glutamine; 3) Amino acids with charged polar R groups (negatively charged at pH 6.0): Aspartic acid, Glutamic acid; 4) Basic amino acids (positively charged at pH 6.0): Lysine, Arginine, Histidine (at pH 6.0). Another grouping may be those amino acids with phenyl groups: Phenylalanine, Tryptophan, and Tyrosine.

The CAR includes a sequence that is at least 90%, at least 95%, at least 98% identical to or identical to the mature amino acid sequence depicted in FIGS. 32-43 (SEQ ID Nos: 29-40), either including or excluding the GMCSFRa signal sequence and either including or excluding the T2A ribosomal skip sequence and the truncated EGFRt). Additional HIV-targeted CAR can be generated by replacing the scFv of any of these CARs with one from FIGS. 3A-3B.

In some cases, the HIV-CAR can be produced using a vector in which the CAR open reading frame is followed by a T2A ribosome skip sequence and a truncated EGFR (EGFRt), which lacks the cytoplasmic signaling tail. In this arrangement, co-expression of EGFRt provides an inert, non-immunogenic surface marker that allows for accurate measurement of gene modified cells, and enables positive selection of gene-modified cells, as well as efficient cell tracking of the therapeutic T cells in vivo following adoptive transfer. Efficiently controlling proliferation to avoid cytokine storm and off-target toxicity is an important hurdle for the success of T cell immunotherapy. The EGFRt incorporated in the HIV-CAR lentiviral vector can act as suicide gene to ablate the CAR-T cells in cases of treatment-related toxicity.

The CAR described herein can be produced by any means known in the art, though preferably it is produced using recombinant DNA techniques. Nucleic acids encoding the several regions of the chimeric receptor can be prepared and assembled into a complete coding sequence by standard techniques of molecular cloning known in the art (genomic library screening, overlapping PCR, primer-assisted ligation, site-directed mutagenesis, etc.) as is convenient. The resulting coding region is preferably inserted into an expression vector and used to transform a suitable expression host cell line, preferably a T lymphocyte cell line, and most preferably an autologous T lymphocyte cell line.

Various T cell subsets isolated from the subject can be transduced with a vector for CAR expression. Central memory T cells are one useful T cell subset. Central memory T cell can be isolated from peripheral blood mononuclear cells (PBMC) by selecting for CD45RO+/CD62L+ cells, using, for example, the CliniMACS® device to immunomagnetically select cells expressing the desired receptors. The cells enriched for central memory T cells can be activated with anti-CD3/CD28, transduced with, for example, a lentiviral vector that directs the expression of an HIV-CAR as well as a non-immunogenic surface marker for in vivo detection, ablation, and potential ex vivo selection. The activated/genetically modified HIV central memory T cells can be expanded in vitro with IL-2/IL-15 and then cryopreserved.

### Construction and Structure of epHIV7 Used for Expression of HIV-Specific CAR

The pHIV7 plasmid is a parent plasmid from which the clinical vectors expressing a HIV-CAR can be derived. The epHIV7 vector used for expression of the CAR was produced from pHIV7 vector (Wang et al. 2011 Blood 118:1255). Importantly, this vector uses the human EFI promoter to drive expression of the CAR. Both the 5′ and 3′ sequences of the vector were derived from pv653RSN as previously derived from the HXBc2 provirus. The polypurine tract DNA flap sequences (cPPT) were derived from HIV-1 strain pNL4-3 from the NIH AIDS Reagent Repository.

Construction of pHIV7 was carried out as follows. Briefly, pv653RSN, containing 653 bp from gag-pol plus 5′ and 3′ long-terminal repeats (LTRs) with an intervening SL3-neomycin phosphotransferase gene (Neo), was subcloned into pBluescript, as follows: In Step 1, the sequences from 5′ LTR to rev-responsive element (RRE) made p5′HIV-1 51, and then the 5′ LTR was modified by removing sequences upstream of the TATA box, and ligated first to a CMV enhancer and then to the SV40 origin of replication (p5′HIV-2). In Step 2, after cloning the 3′ LTR into pBluescript to make p3′HIV-1, a 400-bp deletion in the 3′ LTR enhancer/promoter was made to remove cis-regulatory elements in HIV U3 and form p3′HIV-2. In Step 3, fragments isolated from the p5′HIV-3 and p3′HIV-2 were ligated to make pHIV-3.In Step 4, the p3′HIV-2 was further modified by removing extra upstream HIV sequences to generate p3′HIV-3 and a 600-bp BamHI-SalI fragment containing WPRE was added to p3′HIV-3 to make the p3′HIV-4. In Step 5, the pHIV-3 RRE was reduced in size by PCR and ligated to a 5′ fragment from pHIV-3 (not shown) and to the p3′HIV-4, to make pHIV-6. In Step 6, a 190-bp BglII-BamHI fragment containing the cPPT DNA flap sequence from HIV-1 pNL4-3 (55) was amplified from pNL4-3 and placed between the RRE and the WPRE sequences in pHIV6 to make pHIV-7. This parent plasmid pHIV7-GFP (GFP, green fluorescent protein) was used to package the parent vector using a four-plasmid system.

A packaging signal, psi ψ, is required for efficient packaging of viral genome into the vector. The RRE and WPRE enhance the RNA transcript transport and expression of the transgene. The flap sequence, in combination with WPRE, has been demonstrated to enhance the transduction efficiency of lentiviral vector in mammalian cells.

The helper functions, required for production of the viral vector, are divided into three separate plasmids to reduce the probability of generation of replication competent lentivirus via recombination: 1) pCgp encodes the gag/pol protein required for viral vector assembly; 2) pCMV-Rev2 encodes the Rev protein, which acts on the RRE sequence to assist in the transportation of the viral genome for efficient packaging; and 3) pCMV-G encodes the glycoprotein of the vesiculo-stomatitis virus (VSV), which is required for infectivity of the viral vector.

There is minimal DNA sequence homology between the pHIV7 encoded vector genome and the helper plasmids. The regions of homology include a packaging signal region of approximately 600 nucleotides, located in the gag/pol sequence of the pCgp helper plasmid; a CMV promoter sequence in all three helper plasmids; and a RRE sequence in the helper plasmid pCgp. It is highly improbable that replication competent recombinant virus could be generated due to the homology in these regions, as it would require multiple recombination events. Additionally, any resulting recombinants would be missing the functional LTR and tat sequences required for lentiviral replication.

The CMV promoter was replaced by the EF1α-HTLV promoter (EF1p), and the new plasmid was named epHIV7. The EF1p has 563 bp and was introduced into epHIV7 using NruI and NheI, after the CMV promoter was excised.

The lentiviral genome, excluding gag/pol and rev that are necessary for the pathogenicity of the wild-type virus and are required for productive infection of target cells, has been removed from this system. In addition, epHIV7 vector construct does not contain an intact 3′LTR promoter, so the resulting expressed and reverse transcribed DNA proviral genome in targeted cells will have inactive LTRs. As a result of this design, no HIV-I derived sequences will be transcribed from the provirus and only the therapeutic sequences will be expressed from their respective promoters. The removal of the LTR promoter activity in the SIN vector is expected to significantly reduce the possibility of unintentional activation of host genes.

### CMV-HIV CAR T Cells

T cells specific for CMV and HIV gp120 can be generated using a rapid and efficient method for generating and selecting CMV-specific T cells. The method, which employs IFNγ capture of CMV-specific T cells, consistently and efficiently enriched CMV-specific T cells while preserving the broad spectrum of CMV repertoires. Moreover, the cells remained amenable to gene modification after a brief CMVpp65 stimulation, avoiding the need for CD3/CD28 bead activation prior to transduction. The bulk IFNγ-captured T cells can be engineered with a CAR lentivirus.

Efficient in vivo activation of virus-specific T cells through the TCR demands that viral antigens are processed and presented in a human leukocyte antigen (HLA)-dependent manner. This can be achieved by administering CMV Triplex Vaccine to the subject subsequent to administration of the CMV-CAR T cells.

### Triplex Vaccine

CMV Triplex Vaccine is a recombinant MVA that expresses three CMV antigens, i.e., at least a portion or Immediate-Early Gene-1 (IE1), at least a portion of Immediate-Early Gene-2 (IE2) and at least a portion of pp65. The IE1 antigen and the IE2 antigen can be expressed a fusion protein. Expression of the CMV antigens can be under the control of a modified H5 (mH5) promoter. A CMV Triplex Vaccine is fully described in U.S. Pat. No. 8,580,276 and in Wang et al. (Vaccine 28:1547, 2010)

The CMV Triplex Vaccine can express CMV pp65 and an CMV IE fusion protein (IEfusion). The IEfusion can include an antigenic portion of IE1 (e.g., Exon 4) and an antigenic portion of 1E2 (e.g., Exon 5), wherein the antigenic portions elicit an immune response when expressed by a vaccine.

As explained in U.S. Pat. No. 8,580,276, the CMV Triplex Vaccine includes three of the best recognized antigens in the CD8 subset: pp65, IE1, and IE2. There is no region of homology greater than 5 amino acids between the major exons of both proteins. Individually, both antigens are recognized broadly by almost 70% of the general population (Sylwester et al. 2005). The divergent sequence of both IE1/e4 and IE2/e5 used here predicts an entirely different subset of HLA binding peptides using publicly available Class I and II motif algorithms (Peters and Sette 2007). Human subjects that were evaluated for recognition of both IE1 and IE2 antigens were found in many instances to recognize one or the other but not both. Among the research subjects analyzed, 24% recognized IE2 with or without pp65 to the exclusion of IE1. This result strongly suggests that the recognition elements for both antigens are unique, and by including both of them in the vaccine, the breadth of individuals with disparate HLA types that will recognize and develop an immune response to the vaccine is extended. The fusion of major exons from both antigens achieves the dual goal of reducing the number of separate inserts and eliminating the need for a third insert promoter. The advantages of this approach include placement of all vaccine antigens in one vector, and diminishing the dose of virus needed to attain sufficient immunity simultaneously against all of the included antigens.

### EXAMPLES

### Example 1: HIV-CAR Design

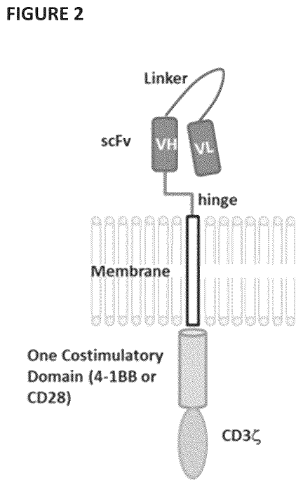

Chimeric antigen receptor (CAR) T cells have been used for control of HIV-1 infection. A schematic drawing depicting a CAR T cell is shown in FIG. 2. A general rationale for the use of CAR T cells for control of HIV-1 infection recognizes that the natural cellular immune response to HIV-1 that limits control of infection is defective. Strategies that require native T-cell receptor-based immunity have failed, most likely due to HIV-1 immune escape mechanisms. The use of ART controls HIV-1 infection and has greatly improved the life of HIV-1 infected persons, although this limits the HIV-1 antigen exposure to the immune system further impacting an effective immune response to the virus. Several broadly neutralizing antibodies (bNAb) against HIV epitopes have been characterized and could be used to construct HIV-targeting CARs [1]. Thus, as described below, we first screened a library of bNAb single chain variable fragments (scFv) for ability to kill HIV gp120-expressing cells and then rescreened candidate CAR genetic constructs for optimization of linkers and costimulatory molecules. With this completed, we then developed a method for production of CMV-specific anti-HIV CAR T cells. Thus, in preclinical studies, we created and tested the following cell products:
CAR T cells specific for several bNAb binding sites in the HIV-1 gp120 protein
CAR T cells optimized for linkers and scFv
Bi-specific CMV-HIV CAR T cells starting from:
   PBMCs collected from CMV-seropositive HIV-seronegative donors; and
   PBMCs collected from CMV-seropositive HIV-seropositive donors.

The overall design for these studies is that a bispecific T-cell response, expanded by a CMV antigen exposure, could be sufficient to markedly reduce the HIV cellular reservoir, as HIV-1 reactivates during a period of analytic treatment interruption (ATI), leading eventually to eradication of HIV from its reservoir.

### Example 2: CAR T Cells Specific for Several bNAb Binding the HIV-1 gp120 Protein

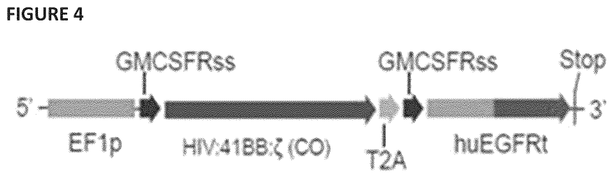

To design the CMV-HIV CAR T cells, the gp120 protein targets of the several bNAbs for which there is genetic sequence information were analyzed. FIGS. 3A-3B shows these targets and the bNAbs. Such bNAbs target epitopes on the HIV-gp120virus spike, the neutralization spectrum is broadest for those which bind to the CD4binding site, the membrane proximal region, and the V3 glycan regions [1-3]. The respective scFv sequences of several of these were cloned into lentivirus (LV) CAR constructs, as depicted in the schematic drawing FIG. 4. These LVs were used to make the first round of candidate HIV-CAR T cells for screening of cytotoxic function vs gp120-expressing CEM cells or 293T cells and for antiviral function vs acute/chronically infected cell lines. Additional CAR constructs using other targets or antibodies can be similarly used. FIGS. 5A-5B shows some sequences.

### Example 3 Selection of HIV-Specific CAR T Cells

Using a previously published method [5] and the lentiviral vector depicted in FIG. 4, studies were performed with a mix of normal donor CD4+ and CD8+ T cells generated to express CARs directed specifically at several binding sites on the gp120 HIV envelope. To prepare the CAR T cells, healthy blood donors were used as well as HIV-infected donors; in addition, several HIV strains and cellular materials were used as targets, as described below.

### Cellular and Viral Materials Used

HIV-1 Strains [source: NIH AIDS repository]

NL4-3—CXCR4- or X4-tropic HIV-1 strain
BaL—CCR5- or R5-tropic HIV-1 strain
89.6—dual tropic HIV-1, molecular clone derived from peripheral blood [6]
LAV—one of the original HIV-1 strains isolated from patient BRU (lymphoadenopathy-
associated virus)

The cell lines used include 8E5 cells, Jurkat cells, ACH-2 cells, 293 T cells, and LCL-OKT3 cells.

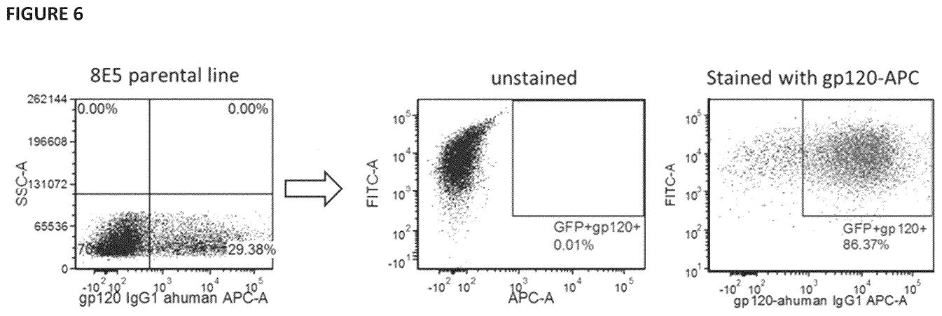

To create a target cell line that expresses the envelope protein HIV-1 gp120, 8E5 cells were used, obtained from ATCC. 8E5 was derived from the T-cell leukemia cell line CCRF-CEM, and has been infected with HIV. 8E5 is a single clone found to have defective provirus, and these cells cannot generate replicative virus but can make most HIV viral proteins. The 8E5 cells were stained with a gp120 recognizing antibody and ˜30% expressed gp120 on the cell surface when evaluated by FACS (FIG. 6; left panel). We transduced these cells with a lentivirus encoding eGFP and the fluorescent firefly luciferase (ffLuc) at an MOI of 10. As shown in FIG. 6, right panel, sorted transduced cells cultured for 14 days showed a significant yield of double positive, GFP+ gp120+ cells, ˜86%. Upon further culture, these cells maintained this dual expression and were used as targets in subsequent CAR T cell functional assays

To create a cell line for in vitro acute HIV infection modeling, Jurkat cells were infected with HIV NL4-3 (MOI=0.01) for 4 hours before co-culture with the CAR T cells. Culture supernatant was collected at days 2, 4, 6, 8, and 12 and evaluated for p24 levels.

To create a model for chronic HIV infection, Jurkat cells were infected with strain NL4-3 HIV at a MOI of 0.1 and then maintained in culture with RPMI media and 10% FBS for more than 4 weeks. This cell line continuously expressed p24 in the supernatant.

Jurkat J1.1 is a cell line latently infected with HIV LAV, in which HIV can be induced with PMA.

ACH-2 is a T cell clone originating from a CEM cell line that was latently infected with HIV-1 LAV; HIV can be induced from ACH-2 using PMA.

Using the same method as shown in FIG. 6 for 8E5 cells, 293 T cells were transduced to express gp120 and either GFP and mCherry.

LCL-OKT3 is a cell line engineered to express a membrane bound OKT3 single chain antibody (OKT3scFv) as described previously [7]. These cells were used as controls.

### Example 4: Screening of CAR Constructs

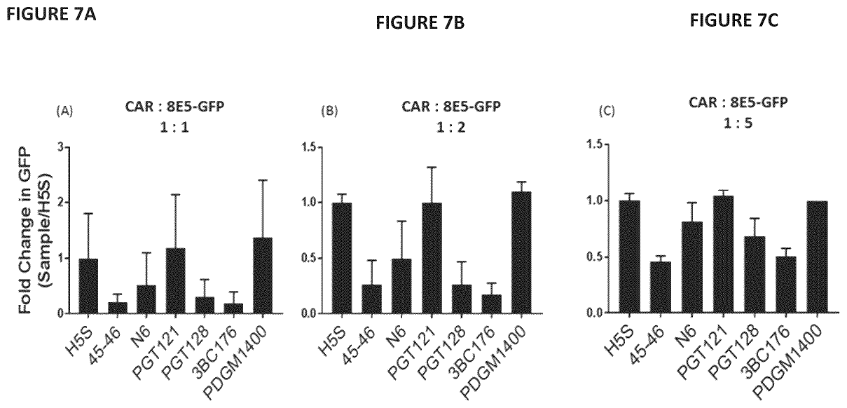

To select the CAR T cell with the highest HIVgp120-specific cytotoxic function, we co-cultured various HIV-CAR T cells at different ratios with 8E5-GFP cells expressing the HIV envelope protein gp120. The CAR T cells targeted the CD4 binding site in gp120 (N6 and 45-46 sites), the V1/2 glycan site (PDGM1400), V3 glycan site (PGT121 and PGT128), or the CD4/V3 sites (3BC176) (FIGS. 3A-3B). A dose dependent effect is observed with most tested CARs (FIGS. 7A-7C), and the CAR constructs targeting the CD4 and CD4/V3 binding sites—45-46, N6, PGT121, and 3BC176 demonstrated the strongest inhibitory effect, shown as fold reduction in GFP in this assay and therefore these underwent further evaluation using virus inhibition assays.

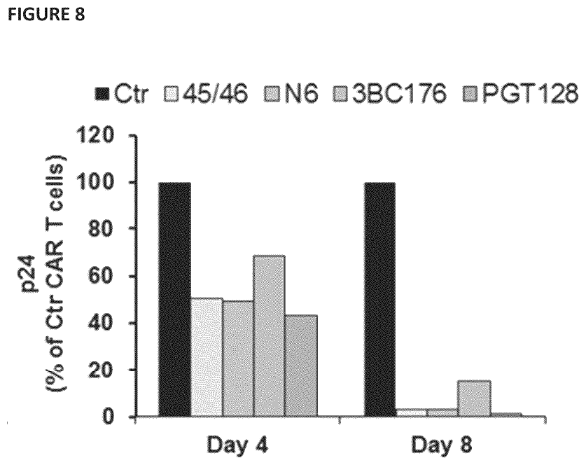

In the first series of antiviral screening assays, CAR constructs targeting the CD4-binding site, the CD4/V3 binding site, and the V3 glycan binding site were evaluated in an in vitro model of chronic HIV-1 infection with HIV (NL4-3) infected Jurkat cells (FIG. 8). The strongest inhibitory effect was observed with CAR constructs targeting the CD4 binding site, N6 and 45-46, and the V3 glycan site, PGT128. The CD4/V3 binding bNAb (3BC176) was eliminated.

Three characteristics of the CARs made from different bNAbs that were used to select the final product. The first of these is the percent of HIV-1 isolates that are neutralized by the parent antibody. The second is the ability of the CARs to both control HIV-1 infection in vitro and kill the gp120-expressing target cells. The third is the ability to make high titer LV for manufacturing of the product. Based on these elements, N6 and 45-46, both targeting the CD4 binding site, were selected for further evaluation.

### Example 5: Optimization of scFv and Linkers for the HIV-CAR Construct

To specifically target and destroy HIV-infected cells, the HIV-CAR constructs require the following: 1) optimization of the scFv as the extracellular antigen recognition domain targeting HIV-1; 2) optimization of spacer or hinge sequences that are used to link the ligand-binding domain to transmembrane and intracellular- signaling domains of the CAR; and 3) best choice of co-stimulatory domain; in this case, the 4-1BB co-stimulatory domain was used, as it has been used successfully in CMV-CD19 CAR T cell studies in animals [5].

To optimize the HIV-CARs, the main focus was on: a) using the recently made available sequences of bNAb to optimize the scFv, and b) comparing the activity of CAR constructs with two linker sequences we had developed for the CD19 CAR T cell projects [8].

CD4-binding site-specific bNAb were used, such as NIH45-46G54W M1 and NIH45-46M2, which were shown to exhibit superior potency and breadth (sequence and specificity shown in Table 4, as derived from the literature [2]; [9]). In addition, the CD4-binding site antibody N6 was evaluated, which had been identified [10]. NIH45-46M2 neutralized 96% of HIV-1 strains in a cross-clade panel and viruses isolated from an HIV-infected individual that are resistant to all other known bNAbs [11]. N6 was found to neutralize 98% of HIV-1 isolates, including 16 of 20 that were resistant to other members of its class. The sequences for these bNAbs are shown in Table 1.

TABLE 4
scFvs of HIV CARs (adapted from Diskin et al, 2013, [9]) (SEQ ID NOS
93-100, respectively)

| | Env | | |
|---|---|---|---|
| Antibody | Specificity | Sequence | |
| NIH45-46 | CD4 Binding | VL | EIVLTQSPATLSLSPGETAIISCRTSQSGSLAWYQQRPGQAPRLVIY |
| WT | Site on gp120 | | SGSTRAAGIPDRFSGRWGADYNLSISNLESGDFGVYYCQQYEF |
| | | | FGQGTKVOVDIK SEQ ID NO: 54 |
| | | VH | QVRLSQSGGQMKKPGESMRLSCRASGYEFLNCPINWIRLAPGR |
| | | | RPEWMGWLKPRGGAVNYARKFQGRVTMTRDVYSDTAFLELRS |
| | | | LTSDDTAVYFCTRGKYCTARDYYNWDFEHWGRGAPVTVSS |
| | | | SEQ ID NO: 101 |
| NIH45-46 | CD4 Binding | VL | EIVLTQSPATLSLSPGETAIISCRTSQSGSLAWYQQRPGQAPRLVIY |
| G54W | Site on gp120 | | SGSTRAAGIPDRFSGSRWGADYNLSISNLESGDFGVYYCQQYEF |
| | | | FGQGTKVQVDIK |
| | | VH | QVRLSQSGGQMKKPGESMRLSCRASGYEFLNCPINWIRLAPGR |
| | | | RPEWMGWLKPRWGAVNYARKFQGRVTMTRDVYSDTAFLELR |
| | | | SLTSDDTAVYFCTRGKYCTARDYYNWDFEHWGRGAPVTVSS |
| NIG45-46 | CD4 Binding | VL | EIVLTQSPATLSLSPGETAIISCRTSQYGSLAWYQQRPGQAPRLVIY |
| M2 | Site on gp120 | | SGSTRAAGIPDRFSGSRWGADYNLSISNLESGDFGVYYCQQYEF |
| | | | FGQGTKVQVDIK |
| | | VH | QVRLSQSGGQMKKPGESMRLSCRASGYEFLNCPINWIRLAPGR |
| | | | RPEWMGWLKPRWGAVNYARKFQGRVTMTRDVYSDTAFLELR |
| | | | SLTSDDTAVYFCTRGKYCTARDYYNWEFEHWGRGAPVTVSS |
| | | | SEQ ID NO: 100 |
| N6 | CD4 Binding | VL | YIHVTQSPSSLSVSIGDRVTINCQTSQGVGSDLHWYQHKPGRAP |
| | Site on gp120 | | KLLIHHTSSVEDGVPSRFSGSGFHTSFNLTISDLQADDIATYYCON |
| | | | LQFFFGRGSRLHIK |
| | | | SEQ ID NO: 99 |
| | | VH | RAHLVQSGTTAMKKPGASVRVSCQTSGYTFTAHILFWFRQAPGR |
| | | | GLEWVGWIKPQYGAVNFGGGFRDRVTLTRDVYREIAYMDIRGL |
| | | | KPDDTAVYYCARDRSYGDSSWALDAWGQGTTVVVSA |

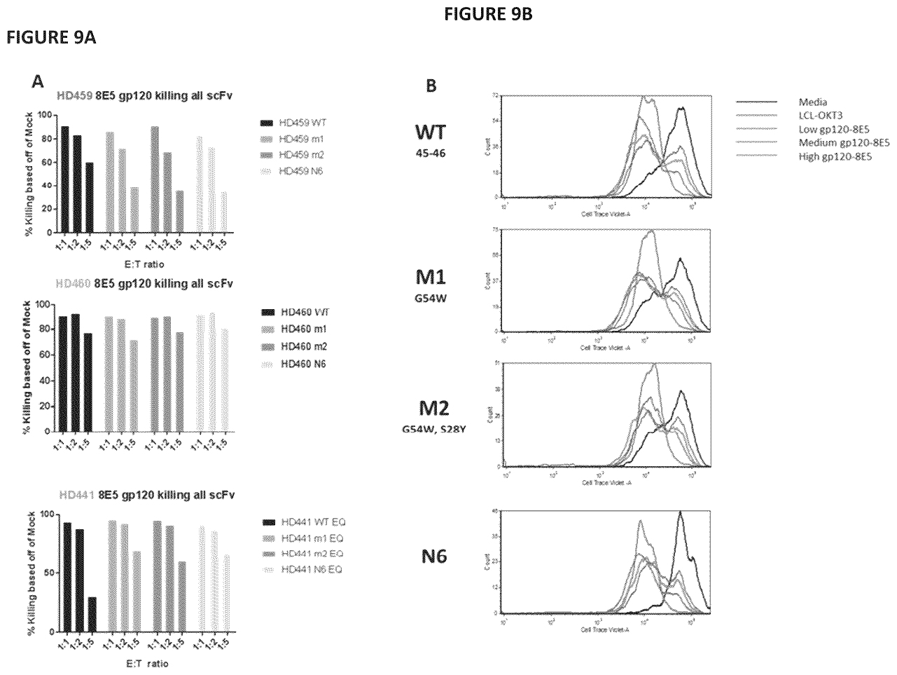

HIV-CAR T cells expressing various scFvs (from WT 45-46, NIH45-46G54W M1 and NIH45-46S28Y M2, as well as N6 bNAb) were generated from three healthy donors (HD459, HD460, and HD441). The cells were co-cultured with gp120^{pos} 8E5-eGFP+ cells at different E:T ratios and 96 hours later, cytotoxicity was evaluated by measuring the % of GFP+ T cells remaining in the co-culture (FIG. 9A).

To track T-cell proliferation, HIV-CAR T cells with different scFvs were stained with 2 μM Cell Trace Violet and co-cultured with varying concentrations of 8E5-gp120 (E:T ratios: 2:1, 1:1, and 0.5:1). After 96 hours in culture, cells were harvested, stained with antibodies specific for T-cell markers and analyzed by flow cytometry (FIG. 9B). The wild-type N6 and 45-46 antibody-based CAR T cells performed slightly better than 45-46 mutant variants, and thus were selected for further testing and development.

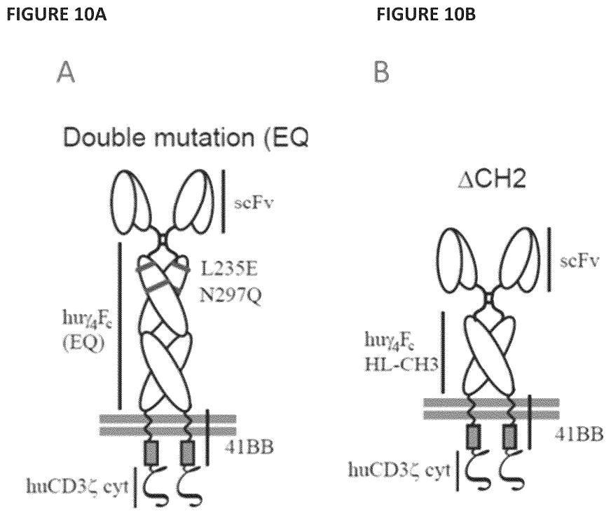

To evaluate whether FcR-mediated interactions play a role in the efficacy of adoptively transferred CAR T cells, two types of CARs were generated: 1) HIV-specific CARs that have been mutated at two sites within the CH2 region (L235E and/or N297Q) of its IgG4 Fc spacer, named EQ mutant (schematic shown in FIG. 10A); and 2) a CAR that has a CH2 deletion in its IgG4 Fc spacer, named ΔCH2 (schematic shown in FIG. 10B).

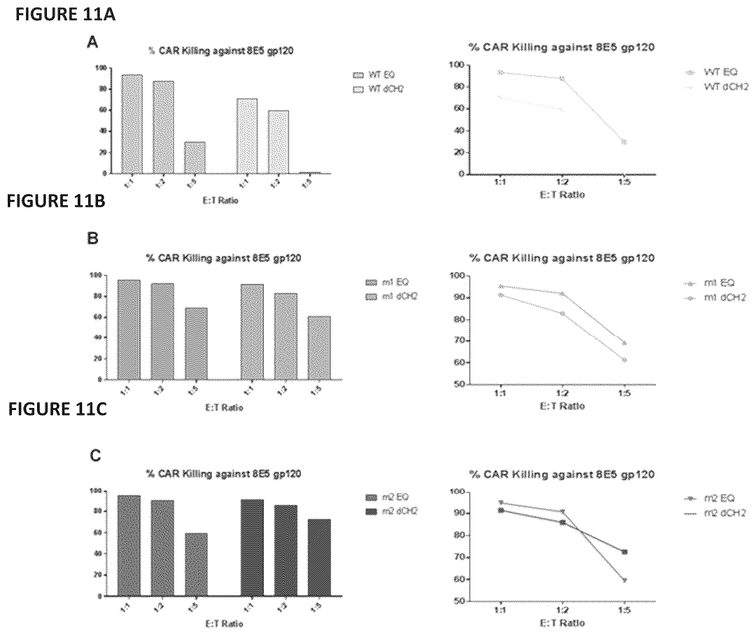

Thus, two linkers of different length were tested to analyze HIV-CAR T cell functionality. The cytotoxicity of HIV-CAR T cells against a gp120-expressing target cells was evaluated, with either an IgG4 (EQ) linker or a shortened IgG4H (ΔCH2) linker [8]. HIV-CAR T cells were co-cultured with the gp120^{pos} 8E5-eGFP+ cells at different E:T ratios. The wild-type and optimized 45-46 CAR constructs were evaluated concomitantly, along with N6. At 96 h post co-culture, cells were stained with antibodies specific for T-cell markers, and cellular toxicity was measured as % GFP change in culture (FIGS. 11A-11C). The CAR construct that contained the EQ linker and WT 45-46 performed better against gp120-expressing target cells.

Based on all linker optimization experimental data, the EQ linker was found to provide improved anti HIV-1 activity for the CAR construct. The scFv for N6 and WT 45-46 had comparable activities, better than the activities of CAR constructs with NIH45-46G54W M1 and NIH45-46S28Y M2. Thus, the final selection was that of EQ linker and N6 or 45-46 scFv to be evaluated as part of CAR constructs in further preclinical development. Subsequent experiments used the HIV CARs that targeted the CD4-binding site.

### Example 6: Antiviral and Cytotoxic Testing of Optimized CAR T Cells

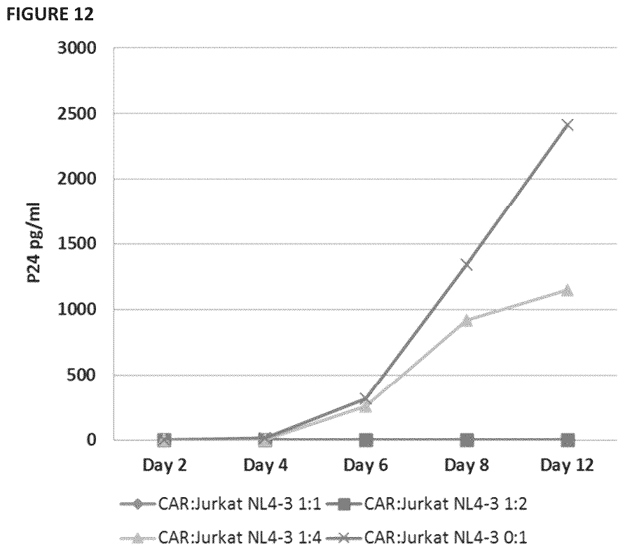

For evaluation of viral inhibition in an acute HIV infection, a model of acute HIV infection in Jurkat cells was developed and used to confirm an antiviral effect of CAR T cells targeting the CD4-binding site of HIV gp120. These HIV-CAR T cells targeting the CD4 binding site and carrying the optimized EQ linker inhibited p24 production in the HIV NL4-3 infected Jurkat cells (FIG. 12), demonstrating their antiviral activity in the acute infection setting in vitro.

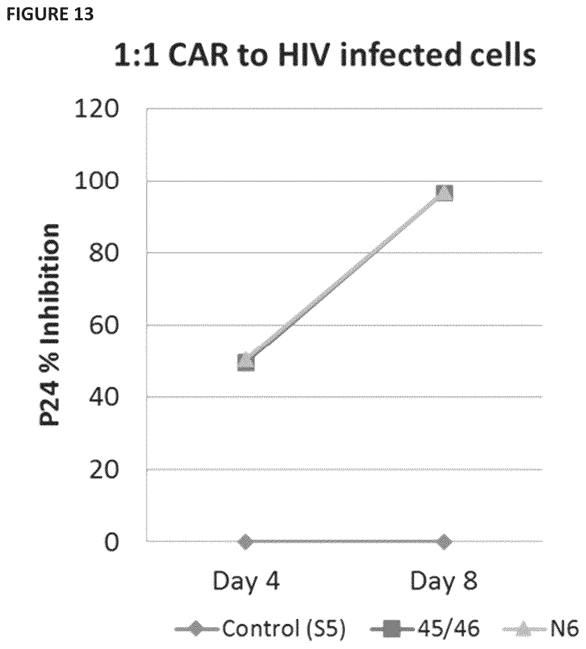

Additional experiments compared the antiviral activity of CAR T cell targeting the CAR binding site of HIV gp120 but derived from bNAbs 45-46 vs N6. CAR T cells were challenged in the setting of chronic infection in Jurkat cells (FIG. 13). Their antiviral activity was found to be equivalent.

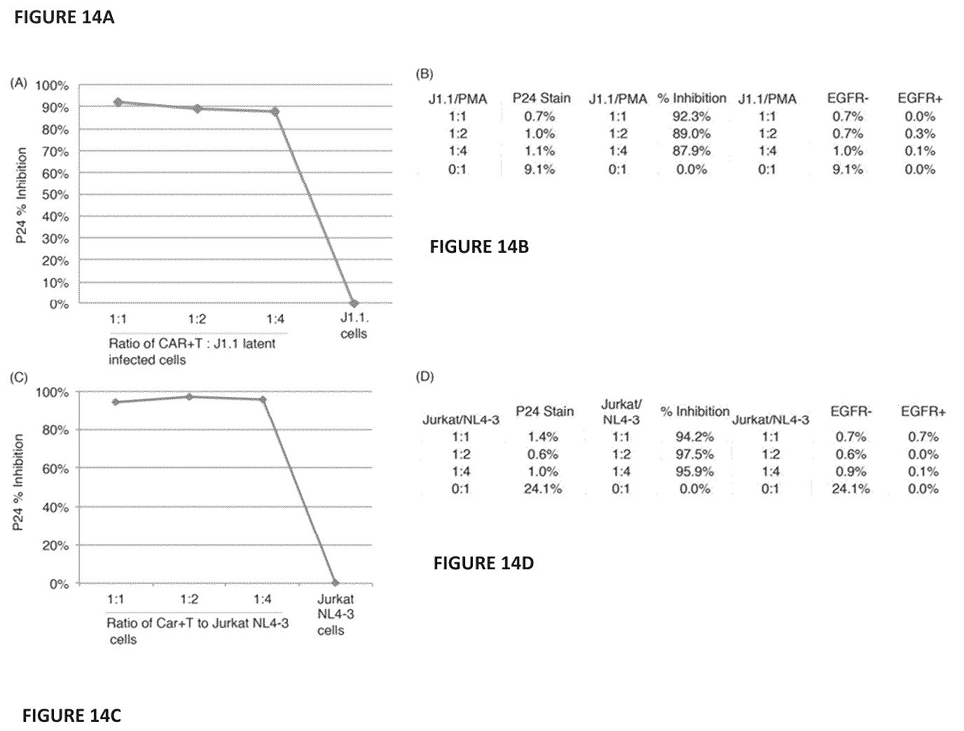

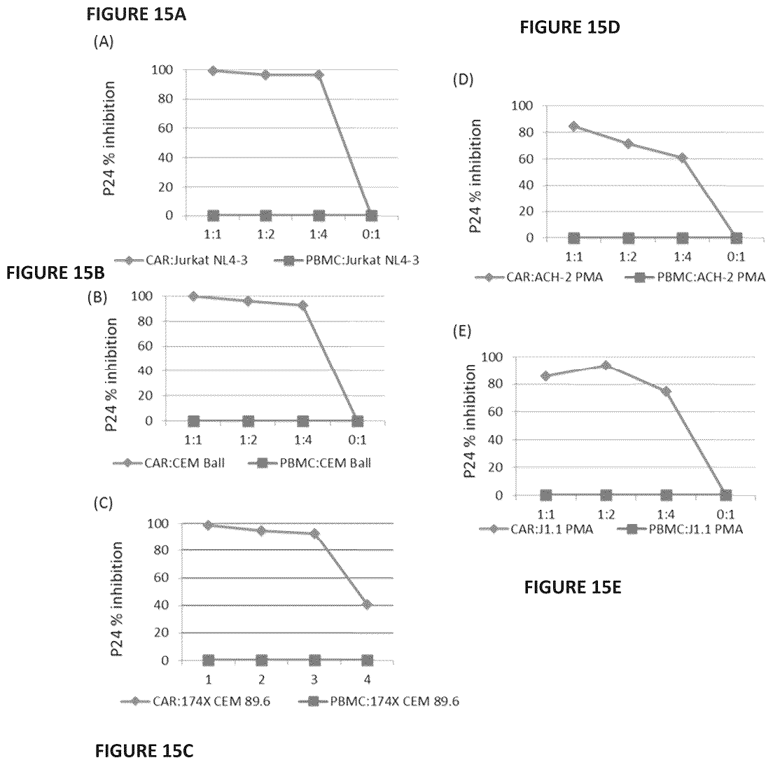

In one experiment, HIV-CAR T cells were generated by transduction with a lentiviral vector containing an EGFR suicide/reporter gene and the broadly neutralizing 45-46 antibody targeted to the CD4 binding site in gp120. In FIG. 14A, various ratios of CAR+ T and J1.1. latently infected jurkat cell were co-cultured and the neutralization of HIV infected cells determined 10 days later. FIG. 14B shows a summary of the CAR+ T/J.1. PMA+ treated cell p24, % inhibition, and CAR vector expression as determined by EGFR expression. In FIG. 15C, various ratios of CAR+ T and Jurkat/NL4-3 infected cells were co-cultured and the neutralization of HIV infected cells determined 10 days later. FIG. 15D shows a summary of the CAR+ T/Jurkat/NL4-3 treated cell p24, % inhibition, and CAR vector expression as determined by EGFR expression.

To evaluate the antiviral activity of these CD4-binding site specific CAR T cells against 5 different HIV-1 strains, the CAR T cells were co-cultured with HIV-infected cells or controls at various ratios, and the inhibition of HIV-infected cells was measured 10 days later (FIGS. 15A-15E). The HIV-CAR T cells were able to inhibit Jurkat cells infected with NL4-3 (T-tropic), CEM cells infected with BaL (M-tropic), 174X CEM cells infected with 89.6 (dual-tropic), ACH-2, a latently HIV LAV infected CEM line, and J1.1, an HIV-1 LAV latently infected Jurkat cell line. Thus, the HIV CAR T cells can inhibit T-tropic, M-tropic, and dual-tropic HIV strains and inhibit latently infected cells that are stimulated in vitro to reactivate HIV infection.

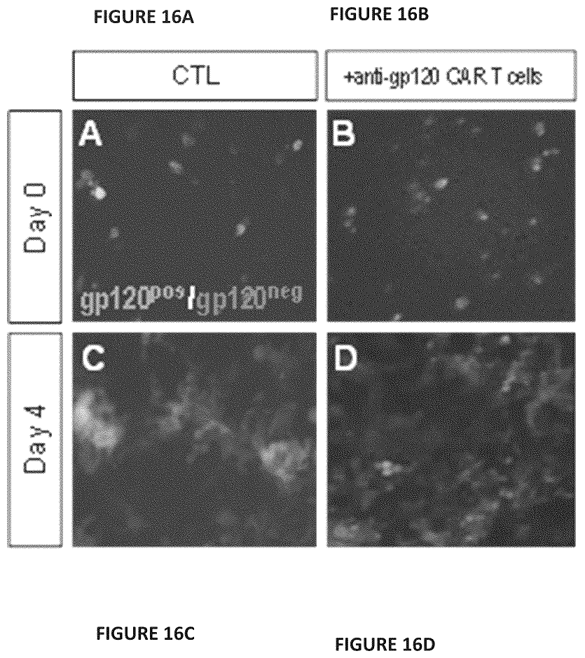

The specificity of HIV-CAR T cell effect in a mixed culture of 293T cells expressing gp120+ [green] and gp120− [red] cells was evaluated (FIGS. 16A-16D). These HIV-specific CAR T cells can eliminate 293T cells that express gp120 in vitro. The gp120+ cells were completely eliminated in 4 days whereas in the absence of CAR T cells, the green and red signals from these gp120+ and gp120− cells remained unchanged (FIG. 16B and FIG. 16D). The effect was specific for gp120, as CD19-specific CAR T cells did not affect gp120-expressing 293T cells.

### Example 7: Development of CAR Cell Swarms for Targeted Eradication of HIV

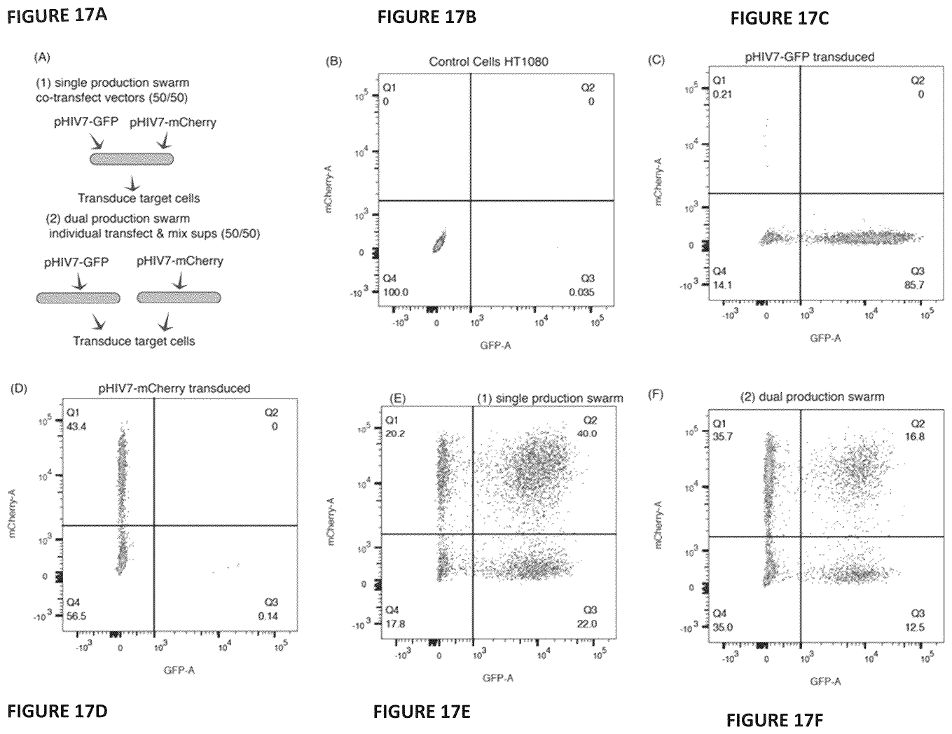

CAR cell swarms were generated as shown in FIGS. 17A-17F.

### Example 8: Combinations of HIV-CAR are More Effective that Single HIV-CAR

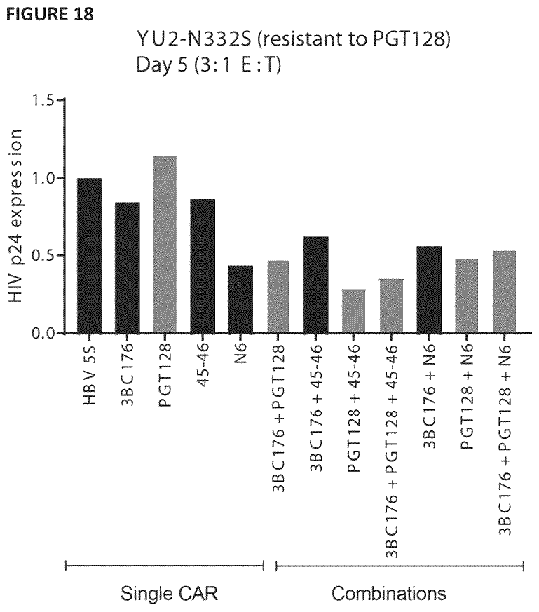

FIG. 18 shows that combinations of two or three distinct HIV-CAR T cells are more cytotoxic than single HIV-CAR T cella against a resistant HIV strain YU2-N332S (resistant to PGT128).

### Example 9: Selection of a Broadly Neutralizing Antibody for Creating a CMV-HIV CAR T Cell

After the identification of 45-46 and N6 as preferred anti-HIV bNAb, cytotoxicity and antigen-driven proliferation of various 45-46 and N6-based CAR T cells were assessed. For 45-46, two mutant variants, (G54W and G54W/S28Y, were tested in addition to a CAR based on wild-type 45-46. All of the CAR were identical outside of the scFv region (CD8 TM, 4-1BB co-stimulatory domain and CD3 zeta). As shown in FIGS. 9A and 9B, 45-46 and N6-based CAR T cells have similar cytotoxicity against gp120^{pos} 8E5-eGFP+ and proliferation in the presence of gp120-positive cells. the titer of the N6 CAR vector was always higher than that of the 45-46 CAR vector during the lentivirus production, and was selected for further development.

### Example 10: CMV-HIV CAR T Cells Target Specifically gp120-Positive Cells in Vitro

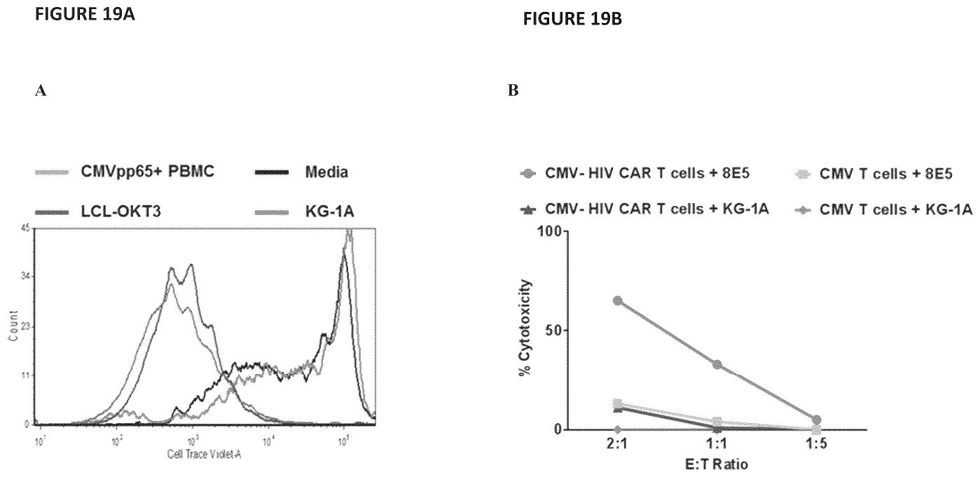

The large-scale selection of CMV-reactive T cells for production of CMV-HIV CAR T cells was based on previously published methods [71]. Briefly, PBMCs from a healthy donor were collected and processed in the CliniMACS Prodigy® system by stimulation of PBMCs with a GMP-grade PepTivator® overlapping CMV pp65 peptide pool, followed by enrichment for CMV-responsive IFNγ+ T cells by using the IFNγ Catchmatrix reagent [Miltenyi Biotec Inc.]. The CAR T cells where expanded for about 3 weeks in vitro and cryopreserved. We confirmed that the CMV-HIV CAR T cells are specifically reactive to CMV and HIV antigens using a proliferation and cytotoxicity assay. CMV-HIV CAR T cells were thawed and selected for EGFR via magnetic selection to generate an EGFR depleted fraction (i.e., CMV-T cells) and an EGFR-enriched fraction (i.e., CMV-HIV CAR T cells). Proliferation assay by dye dilution shows that the CMV-HIV CAR T cells proliferate when co-cultured with CMVpp65+ pulsed autologous PBMCs and LCL-OKT3 (a line engineered to express CD3 receptors and activate T cells), but not when exposed to KG-1A (gp120neg cell line) or media (FIG. 19A). To assess the specific cytotoxicity of the cell product, CMV-HIV CAR T cells or CMV-T cells were co-cultured with KG-1A GFP+ or gp120pos 8E5-eGFP+ cell lines. GFP signal was measured 4 days following co-culture by flow cytometry. CMV-HIV CAR T cells only killed gp120pos cells but not gp120neg cells, while CMV-T cells had little to no toxicity against gp120pos or gp120neg cells (FIG. 19B)

### Example 11: Generation and Characterization of CMV-HIV CAR

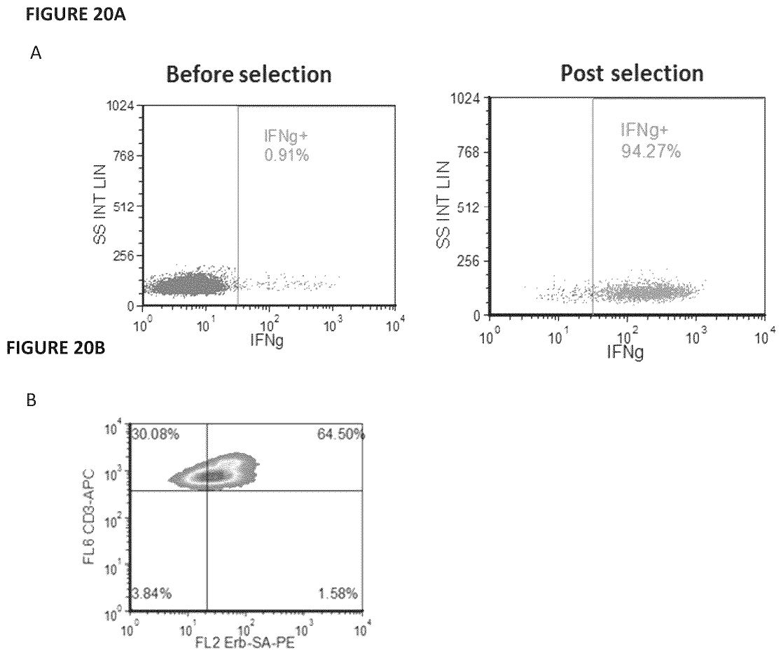

CMV-reactive cells from HIV donors on ART were isolated in the CliniMACS Prodigy® as described before. About 94% of the selected cells were CMV-specific T cells (FIG. 20A). These cells were then transduced with a self-inactivating lentiviral vector encoding either anti-HIV CAR (N6 scFv) or anti-CD19 CAR as control, resulting in ˜65% of CMV-reactive T cells expressing N6-CAR (FIG. 20B) and ˜52% of control CMV-reactive T cells expressing CD19-CAR (data not shown). The CAR T cells were expanded for ˜3 weeks in vitro before functional analysis.

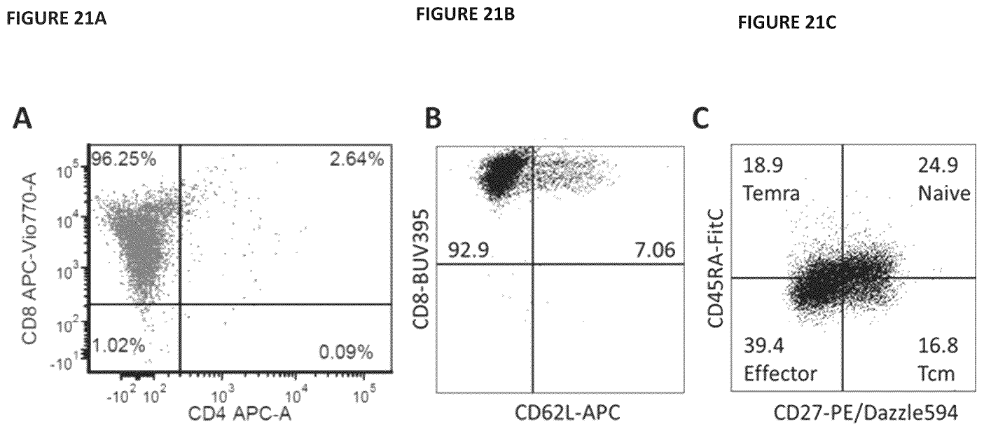

As shown in FIG. 21A, the majority (>96%) of autologous CMV-HIV CAR T cells are CD8+ T cells in the final product from HIV-infected donor. To test whether these CMV-HIV CAR T cells would be stable or terminally differentiate after in vitro expansion, we selected and analyzed EGFR+ cells for the memory/stem marker CD62L (1). About 7% of CMV-HIV CAR T cells express the central memory/stem marker CD62L (FIG. 21B). Since CD62L (a homing molecule) is dynamic, the cells were also assessed for the early stage memory cell marker and co-stimulatory molecule CD27 (TCM and Naïve cells) as well as CD45RA (effector memory T cells, TEMRA). As shown in FIG. 21C, the CMV-reactive CAR T cells contain a diversity of CD8 T cell subpopulations (2). About 42% of the CMV-HIV CAR T cells express the marker CD27 and thus have progenitor/stem properties and/or proliferative capacity (3). Overall, the majority of CMV-HIV CAR T cells have a phenotype conducive to activation and proliferation upon stimulation. We next evaluated these cells for exhaustion markers, since it is known that CAR T cell function is lost as cell exhaustion pathways are activated (4) and that T cells from HIV-infected subjects often express exhaustion markers due to the exposure to HIV (5, 6).

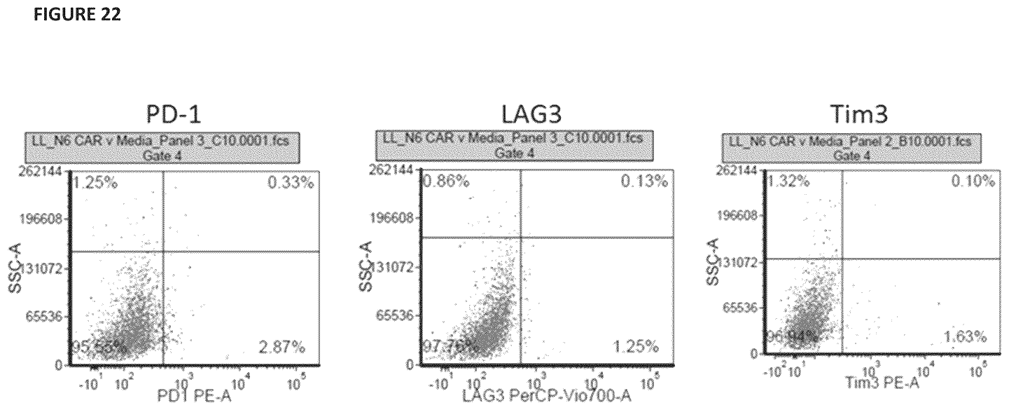

Expression of exhaustion markers (PD-1, LAG3 and Tim3) was analyzed by flow cytometry. After in vitro expansion for 22 days, the CMV-HIV CAR T cells expressed low levels of exhaustion markers (FIG. 22). This observation is consistent with results reported by Gomez-Mora et al. who analyzed cellular immune response of CMV-seropositive HIV-infected individuals with different CD4+ T-cell recovery upon treatment with virologically-suppressive ART (78).

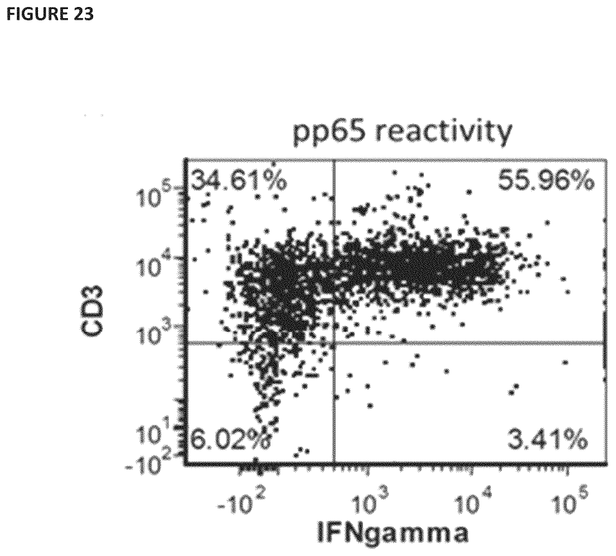

To determine whether CMV-HIV CAR T cells maintain their reactivity to CMV antigens, CMV-HIV CAR T cells were co-cultured with autologous PBMCs pre-stimulated with CMVpp65 peptides followed by antigen recall assay using IFNγ expression. Flow cytometry analyses show that ˜56% of the CD3+ T cells were positive for IFNγ (FIG. 23), indicating a reactivity of the cells to CMV immunogenic epitopes. This results support previous report that T cell activity directed against CMV is preserved in HIV subjects (14).

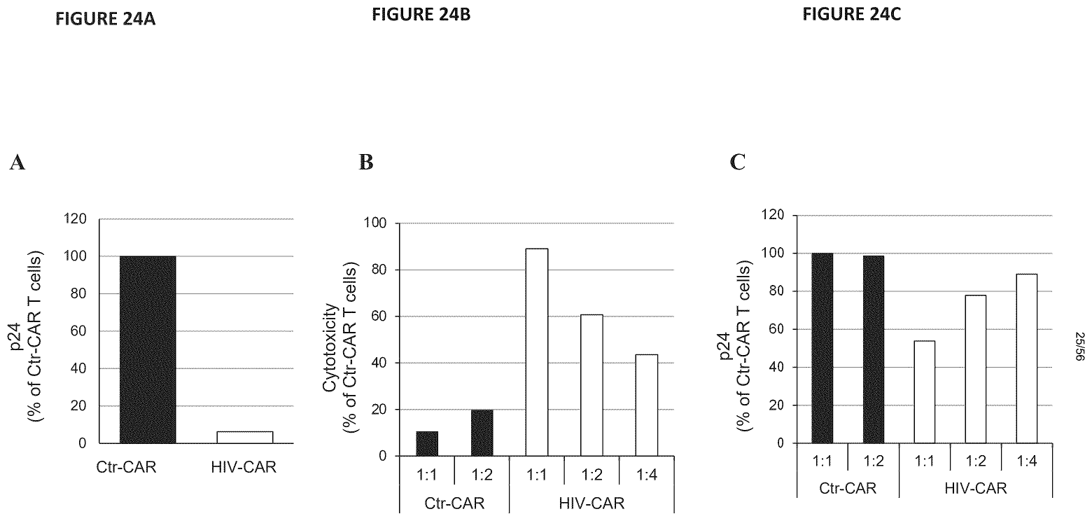

The cytotoxic function of the CMV-HIV CAR T cells was assessed in two in vitro models of HIV: 1) endogenous reactivation of HIV; and 2) Jurkat-GFP+ cells infected with the HIV strain NL4-3 HIV (FIGS. 24A-24C).

In vitro stimulation of CD4+ T cells isolated from HIV donors has been shown to reactivate latent reservoir in culture (79). In the present study, p24 levels were measured by ELISA in the supernatant of CMV-HIV or CMV-CD19 CAR T cells (Ctr-CAR) after 20 days in culture (FIG. 24A). The results demonstrate HIV reactivation in the CMV-CD19 CAR T cell cultures, but not in the CMV-HIV CAR T cell culture medium. Since the source of HIV in these cultures is from the HIV-infected donor, who was aviremic at the time of blood donation, this result shows that the therapeutic product can suppress the HIV reactivation. This study is evidence that autologous CMV-HIV CAR T cells have the potential to suppress HIV reactivation from the HIV reservoir.

In the second study, CMV-HIV or CMV-CD19 CAR T cells (Ctr-CAR) were co-cultured with HIV-infected Jurkat-GFP+ cells. Cytotoxicity was assessed by measuring the percent of GFP+ cells killed after 7 days in culture (FIG. 24B) and the levels of p24 on day 20 (FIG. 24C). These data show a dose-dependent cytotoxic effect and control of HIV replication of the CMV-HIV-but not the CMV-CD19 CAR T cells.

Taken together, the studies in this example demonstrate that 1) can be manufactured in a fully closed semi-automatic system, 2) is predominantly a CD8+ T cell product, 3) remains reactive to CMV stimulation, and 4) is highly potent at controlling HIV reservoir reactivation from the HIV donor in vitro. These data support our therapeutic strategy to combine CMV vaccination with a CAR T cell product to maintain adequate HIV-CAR T cell numbers in HIV-infected individuals during periods of low HIV viremia. We then evaluated our strategy in an animal model of HIV.

### Example 12: Effect of CMV-HIV CAR T Cells on HIV Infection in an Animal Model of HIV

CMV-HIV [N6 scFv] CAR T cells were assessed in an HIV mouse model. These studies assessed CAR T cells expansion and distribution as well as their effect on HIV infection, with or without CMV vaccine.

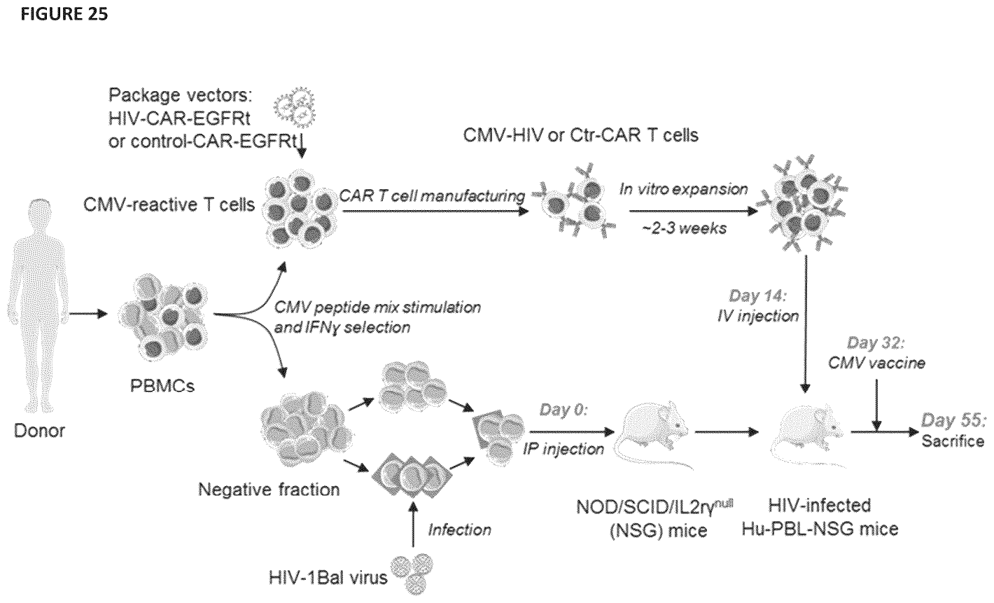

FIG. 25 is a schematic description of the modified HIV-infected Hu-PBL-NSG mouse model. This model is established by engraftment of HIV-challenged donor peripheral blood leukocytes (PBL) in 6-8 week old NSG mouse, an immunodeficient NOD/SCID strain that is devoid of functional murine B, T lymphocytes and NK cells.

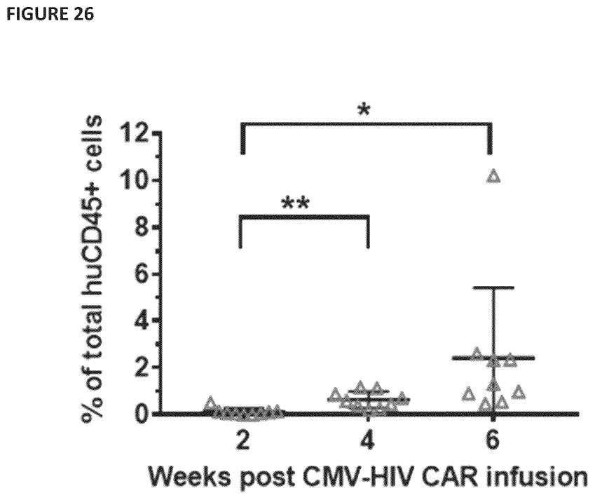

We first explored whether the CMV-HIV CAR T cells from an HIV donor could expand in hu-PBL-NSG mice without vaccination. Briefly, NSG mice were transplanted at Day 0 with PBMCs from an HIV-infected donor on ART. In parallel, CMV-HIV CAR T cells were manufactured from the same HIV-infected donor, as previously described and enriched using EGFR+ selection. Infusing 50,000 CMV-HIV CAR T cells per mouse, CAR T cell expression was followed for 6 weeks. As can be seen in FIG. 26, there was a significant expansion of CMV-HIV CAR T cells in the peripheral blood of HIV-infected mice at 4 and 6 weeks post-CAR T cell infusion, but we did not observe concurrent changes in huCD45, huCD3, huCD4, or huCD8 cells over those same time points (data not shown). This indicates a selective expansion of the CAR T cell, which may be driven by exposure to cell surface-gp120 antigen. This observation is consistent with the putative mechanism for expansion of HIV-CAR T-cells, but it also suggests that expansion in the absence of CMV vaccine may require high levels of viral replication.

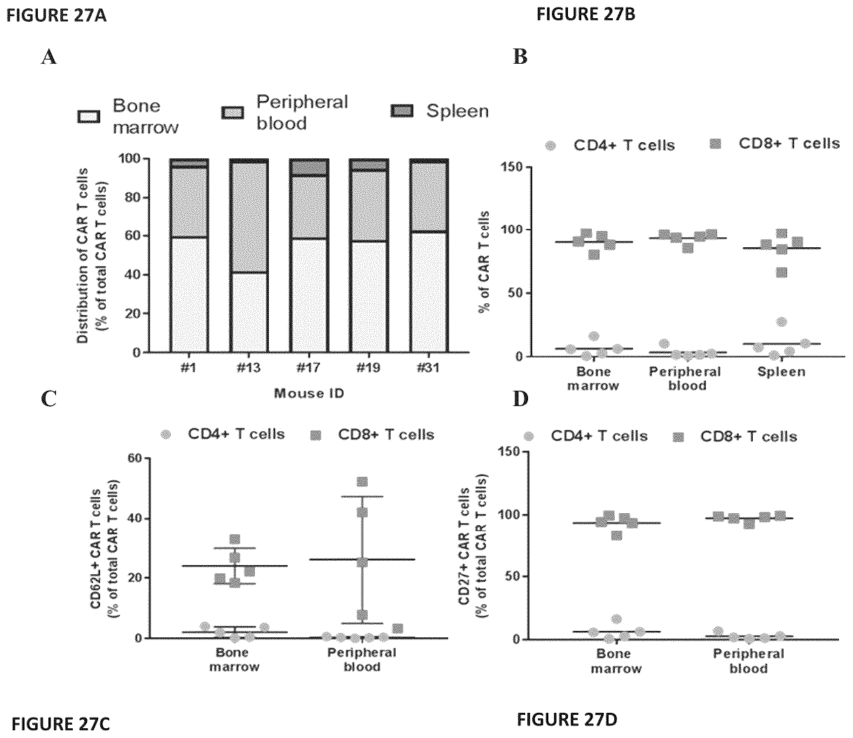

Mice were sacrificed six weeks (Day 55) after CAR T cells infusion and hind quarters (bone marrow), and spleens were removed and processed for flow cytometry analyses to determine CAR T cells' memory state. We found that the CAR T cells were more frequent in the peripheral blood and in the bone marrow, but rare in the spleen (FIG. 27A). As anticipated, the CAR T cells were mostly CD8+ T cells (FIG. 27B). Importantly, CAR T cells still express the early memory cell markers CD62L and CD27 at sacrifice (FIG. 27C and FIG. 27D). These cells were particularly abundant in the bone marrow where central memory T cells are known to reside, and have been shown to improve CAR T cell therapeutic efficiency in the cancer field (80). Thus, these results demonstrate the feasibility of creating CAR T cells from an HIV/AIDS individual that are capable of establishing persistent T cell memory and expansion in vivo.

In a separate experiment using PBMCs from a healthy donor, we analyzed whether the CMV vaccine can enhance expansion of CMV-HIV CAR T cells and what effects this might have on viral replication and protection of uninfected CD4+ T cells. To do so, 1×10⁶ CMV-HIV CAR cells (n=10) or CMV-CS1 CAR T cells (Ctr-CAR T cells, n=10) were infused at Day 14 in the hu-PBL-NSG mouse model. In each cohort, five mice received a vaccination with CMV-peptide coated autologous CMV negative PBMCs on Day 32 and mice were sacrificed on Day 55. Body weight and temperature were measured weekly as a means to monitor toxicity of the therapy. Notably, no differences in body weight or temperature were observed between the experimental cohorts.

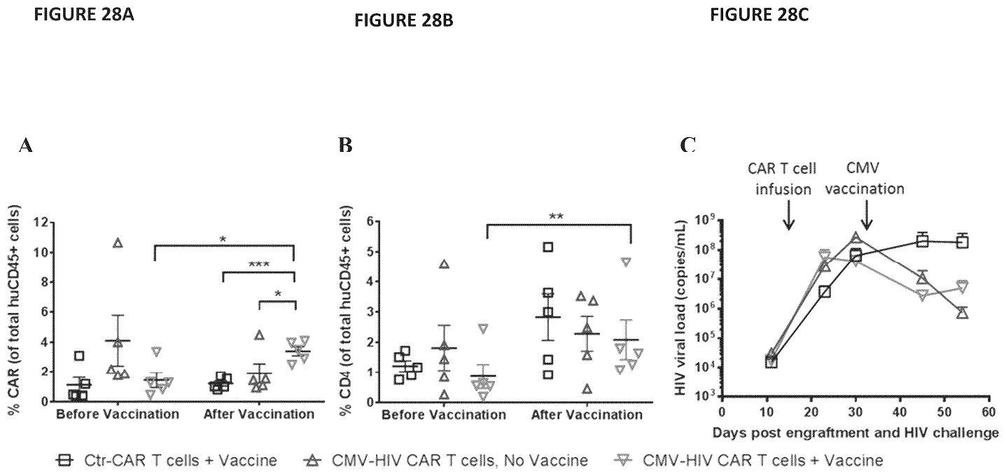

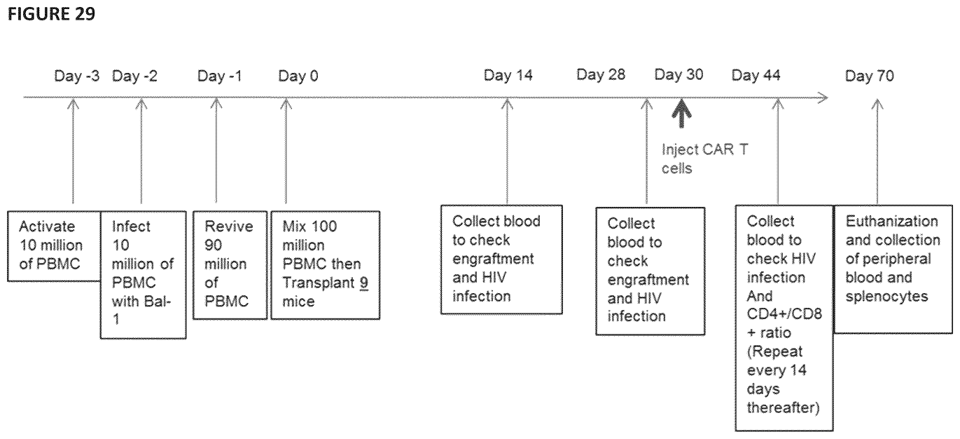

Mice that receive a CMV vaccine exhibited a statistically significant increase of CMV-HIV CAR T cell number two weeks post-vaccination (Day 38 [p<0.05]), consistent with the hypothesis that the vaccine stimulates expansion of CMV-HIV CAR T cells in vivo (FIG. 28A). This increase was also statistically significant relative to the control mice that were CMV-vaccinated (p<0.001). Of note, only the CMV-HIV CARs responded significantly to the vaccine, suggesting some contribution from HIV-CAR working with the CMV signals. Importantly, we observed a statistically significant increase in % huCD4 T cells only in the mice that received CMV-HIV CAR T cells with CMV vaccine (FIG. 28B), indicating that protection of CD4+ T cells occurs even in the absence ART. Finally, we observed a decrease in plasma HIV RNA in mice that received the CMV-HIV CAR T cells (with or without CMV vaccine) compared to those that received the Ctr-CAR T cells with CMV vaccine (74-fold or 18-fold reductions, respectively, at 14 days after CMV vaccination, FIG. 28C).

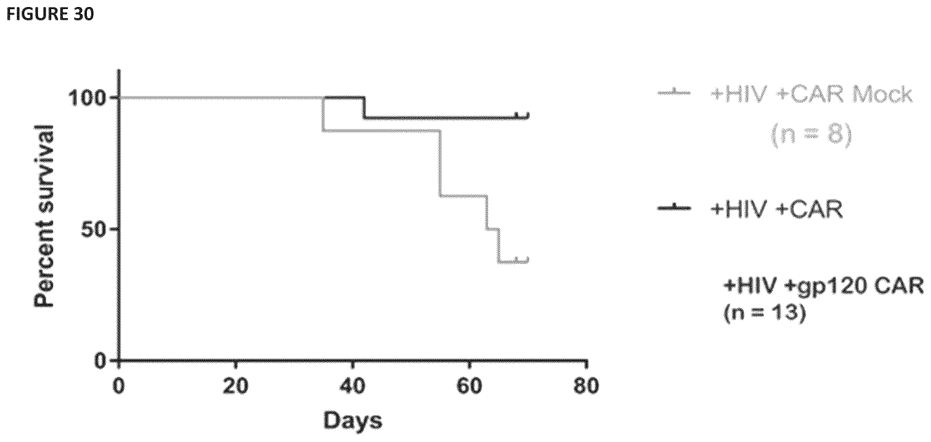

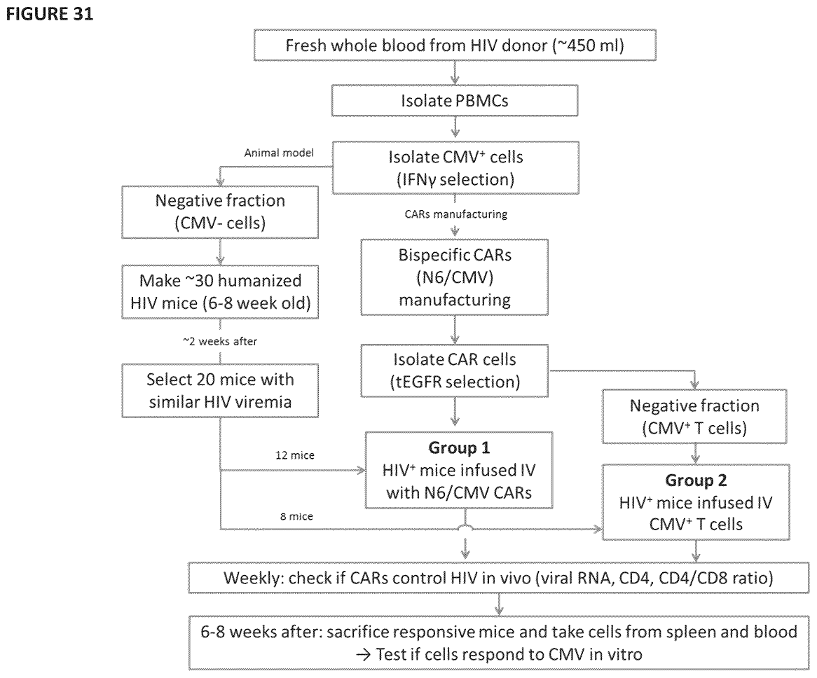

In a systematic study of this model, seven to eight weeks old NSG mice were transplanted with 10×10⁶ PBMC (10⁶ PBMC infected with HIV-1Bal+9×10⁶ uninfected PBMC) by intraperitoneal injection under isoflurane gas anesthesia to establish humanization and HIV infection. Mice that exhibited engraftment (>10% hCD45+ human cells in peripheral blood) and HIV infection (>10⁴ viral copies per ml of blood) were treated with an IV infusion of 10⁶ anti-gp120 CAR T cells (45/46 anti-HIV construct) or a mock lentiviral vector at 4 weeks post transplantation. Mice were monitored for a total of 10 weeks for signs of declining health (hunch and ruffled hair coat, lethargy, dehydration, weight loss) and terminal disease. Mice that were moribund were euthanized and date of death was recorded. Statistical analysis was performed by log rank test, which yielded P=0.012. The Kaplan-Meier survival curve shown in FIG. 30 demonstrates the significantly improved survival (p=0.012) of the mice infected with HIV and which received the gp120+ CAR T cells.

In this study, the anti-HIV effect, 1-2 log reduction in p24 levels, was disproportional to the survival data.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

ADDIN EN.REFLIST 1. V. Golubovskaya, L. Wu, Different Subsets of T Cells, Memory, Effector Functions, and CAR-T Immunotherapy. Cancers 8, (Mar. 15, 2016).
2. A. Larbi, T. Fulop, From “truly naive” to “exhausted senescent” T cells: when markers predict functionality. Cytometry A 85, 25 (January 2014).
3. L. Gattinoni, C. A. Klebanoff, N. P. Restifo, Paths to stemness: building the ultimate antitumour T cell. Nat Rev Cancer 12, 671 (October 2012).
4. H. E. Ghoneim, A. E. Zamora, P. G. Thomas, B. A. Youngblood, Cell-Intrinsic Barriers of T Cell-Based Immunotherapy. Trends Mol Med 22, 1000 (December 2016).
5. C. L. Day et al., PD-1 expression on HIV-specific T cells is associated with T-cell exhaustion and disease progression. Nature 443, 350 (Sep. 21, 2006).
6. L. Trautmann et al., Upregulation of PD-1 expression on HIV-specific CD8+ T cells leads to reversible immune dysfunction. Nat. Med. 12, 1198 (October 2006).

What is claimed is:

1. A human T cell expressing: (a) a T cell receptor (TCR) that is activated by binding to a CMV antigen; and (b) a chimeric antigen receptor (CAR) comprising an antigen-binding domain that binds to an epitope on HIV gp120, a spacer, a transmembrane domain, a co-stimulatory domain, and a CD3 zeta signaling domain.

2. The human T cell of claim 1, wherein the antigen-binding domain is a scFv comprising a heavy chain variable region and a light chain variable region.

3. The human T cell of claim 2, wherein the heavy chain variable region has an amino acid sequence as shown in FIG. 5, and wherein the light chain variable region has an amino acid sequence that is the corresponding light chain variable region sequence shown in FIG. 5.

4. The human T cell of claim 1, wherein the transmembrane domain is a CD4, CD8 or CD28 transmembrane domain.

5. The human T cell of claim 1, wherein the co-stimulatory domain is a 4-1BB or CD28 co-stimulatory domain.

6. The human T cell of claim 1, expressing further a second CAR,
wherein the first CAR comprises a first antigen-binding domain, a first spacer, a first transmembrane domain, a first co-stimulatory domain, and a first intracellular signaling domain,
wherein the second CAR comprises a second antigen-binding domain, a second spacer, a second transmembrane domain, a second co-stimulatory domain, and a second intracellular signaling domain,
wherein the first antigen-binding domain is different from the second antigen-binding domain, and
wherein the first antigen-binding domain binds to a first epitope on HIV gp120, and the second antigen-binding domain binds to a second epitope on HIV gp120, wherein the second epitope is the same as or different from the first epitope.

7. The T cell of claim 6, wherein the first antigen-binding domain comprises a first scFv, and the second antigen-binding domain comprises a second scFv, wherein the amino acid sequence of the first scFv is different from the amino acid sequence of the second scFv.

8. A method for treating HIV comprising administering to a patient in need thereof a population of human T cells comprising T cells expressing CAR targeted to two or more HIV gp120 epitopes.

9. The method of claim 8, wherein the chimeric antigen receptor comprises: a HIV scFv; a spacer region; a CD4, CD8 or CD28 transmembrane domain; a 4-IBB of CD28 co-signaling domain; and a CD3 ζ signaling domain.

10. The method of claim 8, wherein the T cells express a T cell receptor specific for a cytomegalovirus (CMV) antigen.

11. The method of claim 8, wherein the population of human T cells comprising T cells expressing CAR targeted to three or more HIV gp120 epitopes.

12. The method of claim 8, wherein population of human T cells comprising T cells expressing CAR targeted to four or more HIV gp120 epitopes.

13. The method of claim 8, wherein the population of human T cells comprising T cells expressing CAR targeted to six or more HIV gp120 epitopes.

14. The method of claim 8, wherein the patient is not being administered anti-retroviral therapy at the time of administration.

15. The method of claim 8, wherein the patient is further administered a T cell antigen.

16. The method of claim 8, wherein the chimeric antigen receptor comprises an amino acid sequence identical to an amino acid sequence selected from: SEQ ID NOs: 29-40 or 41-53.

17. The method of claim 8, further comprising: administering to the patient a viral vector encoding: (i) CMV pp65 and (ii) a fusion protein comprising exon 4 of CMV protein IE1 (e4) and exon 5 of CMV protein IE2 (e5) either prior to or subsequent to administering the composition comprising a population of T cells to the patient.

18. The method of claim 8, wherein the T cells are allogenic to the patient.

19. The method of claim 8, wherein the T cells are engineered autologous T cells.

20. The method of claim 8, wherein the patient is treated to activate HIV-1 provirus before or after administration of the population of T cells.

21. The method of any of claims 8-20, wherein the T cells express a T cell receptor specific for a cytomegalovirus (CMV) antigen and are expanded in the presence of a T cell antigen before administration to the patient.

22. The method of claim 21, wherein the CMV antigen is a CMV peptide or a CMV vaccine.

23. The method of claim 21, wherein the expansion is in the presence of any HIV antigen and a CMV antigen.

24. The method of claim 21, wherein the expansion is in the absence of an HIV antigen.

25. A method of treating HIV in an HIV infected subject, comprising:
obtaining a population of cells comprising PBMC from the subject and treating the cells to obtain a subpopulation of T cells that express a TCR that binds to an CMV antigen;
treating the subpopulation of cells to introduce a vector encoding a CAR that binds to an epitope on HIV gp120, thereby creating a population of bi-specific T cells expressing a TCR that binds to a CMV antigen and a CAR that binds to an epitope on HIV gp120;
expanding the population of bi-specific T cells; and
administering the expanded bi-specific T cells to the subject.

26. The method of claim 25 further comprising administering a CMV antigen to the subject prior to obtaining a population of cells comprising PBMC from the subject.

27. The method of claim 26, wherein a CMV antigen is administered to the patient before, in conjunction with or after administering the expanded bi-specific T cells to the subject.

28. The method of claim 25, 26 or 27, wherein the CAR comprises an antigen-binding domain that binds to an epitope on HIV gp120, a spacer, a transmembrane domain, a co-stimulatory domain, and a CD3 zeta signaling domain.

29. The method of claim 28, wherein the antigen-binding domain is a scFv comprising a heavy chain variable region and a light chain variable region.

30. The method of claim 29, wherein the heavy chain variable region has an amino acid sequence as shown in FIG. 5, and wherein the light chain variable region has an amino acid sequence that is the corresponding light chain variable region sequence shown in FIG. 5.

31. The method of claim 28, wherein the transmembrane domain is a CD4, CD8 or CD28 transmembrane domain.

32. The method of claim 28, wherein the co-stimulatory domain is a 4-1BB or CD28 co-stimulatory domain.

33. The method of any of claim 25-32, wherein the CAR comprises the amino acid sequence of any of SEQ ID NOS: 20-40 with up to 10 single amino acids substitutions.

34. The method of any of claim 25-32, wherein the CAR comprises the amino acid sequence of any of SEQ ID NOS: 20-40 with up to 5 single amino acids substitutions.

35. The method of any of claim 25-32, wherein the CAR comprises the amino acid sequence of any of SEQ ID NOS: 20-40 with up to 10 single amino acids substitutions.

36. The method of any of claims 25-35, wherein the T cells express a T cell receptor specific for a cytomegalovirus (CMV) antigen and are expanded in the presence of a T cell antigen before administration to the patient.

37. The method of claim 36, wherein the CMV antigen is a CMV peptide or a CMV vaccine.

38. The method of any of claims 25-35, wherein the expansion is in the presence of any HIV antigen and a CMV antigen.

39. The method of claims 25-35, wherein the expansion is in the absence of an HIV antigen.

40. A method comprising:
obtaining a population of PBMC comprising T cells express a TCR that binds to a CMV antigen;
exposing the population of PBMC to a CMV antigen;
treating the exposed cells to isolated a subpopulation of cells enriched for activated cells expressing a TCR that binds to a CMV antigen;
treating the subpopulation of cells to introduce a vector encoding a CAR that binds to an epitope on HIV gp120, thereby creating a population of bi-specific T cells expressing a TCR that binds to a CMV antigen and a CAR binds to an epitope on HIV gp120.

41. The method of claim 40, wherein the CAR comprises an antigen-binding domain that binds to an epitope on HIV gp120, a spacer, a transmembrane domain, a co-stimulatory domain, and a CD3 zeta signaling domain.

42. The method of claim 41, wherein the antigen-binding domain is a scFv comprising a heavy chain variable region and a light chain variable region.

43. The method of claim 41, wherein the heavy chain variable region has an amino acid sequence as shown in FIG. 5, and wherein the light chain variable region has an amino acid sequence that is the corresponding light chain variable region sequence shown in FIG. 5.

44. The method of claim 41, wherein the transmembrane domain is a CD4, CD8 or CD28 transmembrane domain.

45. The method of claim 41, wherein the co-stimulatory domain is a 4-1BB or CD28 co-stimulatory domain.

46. The method of any of claim 41-45, wherein the CAR comprises the amino acid sequence of any of SEQ ID NOS: 20-40 with up to 10 single amino acids substitutions.

47. The method of any of claim 41-45, wherein the CAR comprises the amino acid sequence of any of SEQ ID NOS: 20-40 with up to 5 single amino acids substitutions.

48. The method of any of claim 41-45, wherein the CAR comprises the amino acid sequence of any of SEQ ID NOS: 20-40 with up to 10 single amino acids substitutions.

49. The method of any of claims 41-48, wherein the T cells express a T cell receptor specific for a cytomegalovirus (CMV) antigen and are expanded in the presence of a T cell antigen before administration to the patient.

50. The method of claim 49, wherein the CMV antigen is a CMV peptide or a CMV vaccine.

51. The method of any of claims 41-48, wherein the expansion is in the presence of any HIV antigen and a CMV antigen.

52. The method of claims 41-48, wherein the expansion is in the absence of an HIV antigen.
